(19) United States
(12) Patent Application Publication
(10) Pub. No.: US 2025/0296934 A1
(43) Pub. Date: Sep. 25, 2025

(54) AZEPINOINDOLES AND METHODS OF PREPARATION THEREOF

(71) Applicant: BRIGHT MINDS BIOSCIENCES INC., Vancouver (CA)

(72) Inventors: Alan KOZIKOWSKI, Wallace, MI (US); Werner TUECKMANTEL, Fitchburg, WI (US)

(21) Appl. No.: 18/863,516

(22) PCT Filed: May 2, 2023
(86) PCT No.: PCT/CA2023/050595

§ 371 Date: Nov. 6, 2024

Related U.S. Application Data
(60) Provisional application No. 63/338,889, filed on May 6, 2022.

(51) Int. Cl. C07D 487/04; A61K 31/55; C07D 487/18

(52) U.S. Cl. CPC C07D 487/04; A61K 31/55; C07D 487/18

(57) ABSTRACT

This disclosure relates to heterocyclic compounds of Formula (I). As contemplated herein, heterocyclic compounds of Formula (I) may be used for the treatment of neuropsychiatric, and neurodegenerative, neuroinflammatory and pain disorders including depression, as well as tobacco, opiate, and cocaine addiction, alcoholism, post-traumatic stress disorder (PTSD), and neuropathic pain syndromes including cluster headaches and chemotherapy induced peripheral neuropathy.

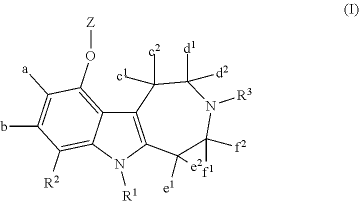

### TECHNICAL FIELD

The present disclosure relates to heterocyclic compounds and methods of preparing the same. The present disclosure also relates to uses of azepinoindoles as selective agents at serotonin receptors.

### BACKGROUND

Psilocybin is a naturally occurring psychedelic compound produced by more than 200 species of mushrooms collectively known as “psilocybin mushrooms”. As a prodrug, psilocybin is quickly metabolized by the body to generate the bioactive compound psilocin, which has mind-altering effects not unlike those produced by other psychedelics such as lysergic acid diethylamide (LSD), mescaline, and N,N-dimethyltryptamine (DMT). These effects include, inter alia, euphoria, visual and mental hallucinations, changes in perception, a distorted sense of time, and spiritual experiences, and can also include possible adverse reactions such as nausea and panic attacks. For reference, the chemical structure of psilocin is provided in FIG. 1 herein.

As agonists of the 5-HT_{2A} and 5-HT_{2C} receptors, psilocybin and psilocin have been recognized for their therapeutic potential. Since 5-HT_{2A} receptor activation appears to increase locomotor activity, whereas 5-HT_{2C} receptor activation appears to decrease locomotor activity, compounds possessing varying degrees of 5-HT_{2A} and 5-HT_{2C} activity will show varying levels of psychedelic activity (Halberstadt A L, van der Heijden I, Ruderman M A, Risbrough V B, Gingrich J A. Geyer M A, Powell S B, Neuropsychopharmacology, 2009, 34 (8): 1958-67). While psilocybin, along with other psychedelic drugs, were explored more than 60 years ago by Hofmann and co-workers at Sandoz (see for example, Hofmann, A., Troxler, F. U.S. Pat. Nos. 3,075,992; 3,078,214), clinical investigations into these drugs waned substantially by the early 1970s-particularly after these drugs were placed on Schedule 1 of the Controlled Substance Act in the United States of America. Notwithstanding their listing as controlled substances in certain jurisdictions however, research into psilocybin and other psychedelic drugs never fully stopped, and recent clinical investigations have led to a revived interest in the potential application of psychedelic drugs (including psilocybin) in evolving medical areas, such as the treatment of central nervous system (CNS) diseases. CNS diseases include both difficult-to-treat mental health disorders (Daniel J, Haberman M. Clinical potential of psilocybin as a treatment for mental health conditions. Ment. Health Clin. 2017, 7 (1), 24-8), such as treatment resistant depression or drug resistant depression, and neurological disorders such as cluster headaches.

While psilocybin has recognized therapeutic potential for treating certain CNS diseases and disorders, it is also recognized as a 5-HT_{2B} receptor agonist and is therefore cardiotoxic. As such, there is an unmet need for safer drugs and analogs of psilocybin and psilocin that maintain 5-HT_{2A} receptor agonist activity but that lack cardiotoxic 5-HT_{2B} agonist activity; furthermore, and at least in some instances, there is an unmet need for safer drugs that maintain 5-HT_{2A} receptor agonist activity but that lack cardiotoxic 5-HT_{2B} agonist activity.

### SUMMARY

The present disclosure relates to indole compounds, and in particular to compounds that belong to the azepinoindole class of compounds, that exhibit 5-HT_{2A} receptor agonist activity while exhibiting low 5HT_{2B} receptor agonist activity. In at least some cases, such compounds show selectivity for the 5-HT_{2A} receptor over the 5-HT_{2C} receptor. The compounds disclosed herein may be useful in the treatment of depression including major depressive disorder, drug resistant depression, and psychotic depression, addiction including alcoholism, tobacco addiction, cocaine addiction, and opioid addiction, pain indications including neuropathic pain, pain from chemotherapy associated neuropathy, phantom limb pain and fibromyalgia, inflammation (including chronic and acute), eating disorders including anorexia, autism, cluster headaches, migraines, dementia including Alzheimer's dementia, Parkinson's disease dementia, and Lewy body dementia, post-traumatic stress disorder, emotional distress associated with cancer, Fragile-X syndrome, autism spectrum disorder, bipolar disease, obsessive compulsive disease, Rett syndrome, and other CNS disorders.

According to a part of the present disclosure, there are chemical entities of Formula I,

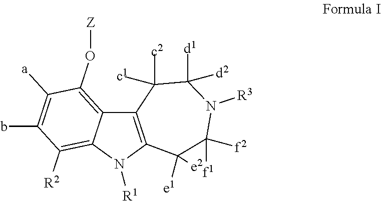

wherein R¹, R², R³, a, b, c¹, c², d¹, d², e¹, e², f¹, f², and Z are defined hereinafter.

The chemical entities of Formula I are 5-HT_{2A} receptor agonists with selectivity over the 5-HT_{2B} subtype. Chemical entities of Formula I, and pharmaceutically acceptable compositions thereof, are potentially useful for treating a variety of diseases and disorders associated with 5-HT2A receptor agonism. Such diseases and disorders include those described herein.

This summary does not necessarily describe the entire scope of all aspects of the disclosure. Other aspects, features and advantages will be apparent to those of ordinary skill in the art upon review of the following description of specific embodiments.

### BRIEF DESCRIPTION OF DRAWINGS

In the accompanying drawings, which illustrate one or more embodiments:

FIG. 1 depicts the chemical structure of psilocin.

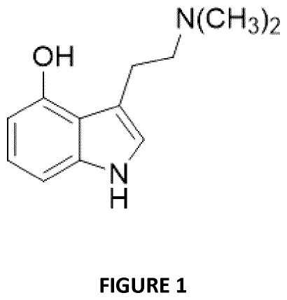

FIG. 2 depicts the chemical structure of compounds of Formula I.

FIG. 3 is a graph depicting an HTR over time per dose (mg/kg) for Compound 1 (as described herein), as administered to mice.

### DETAILED DESCRIPTION

Directional terms such as “top,” “bottom,” “upwards,” “downwards,” “vertically,” and “laterally” are used in the following description for the purpose of providing relative reference only, and are not intended to suggest any limitations on how any article is to be positioned during use, or to be mounted in an assembly or relative to an environment. The use of the word “a” or “an” when used herein in conjunction with the term “comprising” may mean “one,” but it is also consistent with the meaning of “one or more,” “at least one” and “one or more than one.” Any element expressed in the singular form also encompasses its plural form. Any element expressed in the plural form also encompasses its singular form. The term “plurality” as used herein means more than one, for example, two or more, three or more, four or more, and the like.

As used herein and unless otherwise specified, the term “about”, when used to describe a recited value, means within 10% of the recited value.

As used herein and unless otherwise specified, the term “alkenyl” refers to a substituted or unsubstituted, linear or branched, univalent hydrocarbon chain having at least two carbon atoms and at least one carbon-carbon (CC) double bond. Examples of alkenyl groups include allyl, vinyl, 2-propenyl, crotyl, 2-isopentenyl, 1,3-butadien-2-yl, 2,4-pentadien-1-yl, 1,4-pentadien-3-yl, and the like.

As used herein and unless otherwise specified, the term “alkoxy”, used alone or as part of a larger moiety, refers to the groups-O-alkyl and —O-cycloalkyl. As used herein and unless otherwise specified, the term “substituted alkoxy”, used alone or as part of a larger moiety, refers to the groups-O-(substituted alkyl) and —O-(substituted cycloalkyl).

As used herein and unless otherwise specified, the term “alkyl”, used alone or as part of a larger moiety, means a substituted or unsubstituted, linear or branched, univalent hydrocarbon chain that is completely saturated. Unless otherwise specified, an alkyl group contains 1 to 7 carbon atoms (“C₁-C₇ alkyl”). For example, in some embodiments, alkyl groups contain 1 to 6 carbon atoms (“C₁-C₆ alkyl”); in some embodiments, alkyl groups contain 1 to 5 carbon atoms (“C₁-C₅ alkyl”); in some embodiments, alkyl groups contain 1 to 4 carbon atoms (“C₁-C₄ alkyl”, alternatively “lower alkyl”); and in some embodiments, alkyl groups contain 3 to 7 carbon atoms (“C₃-C₇ alkyl”). Non-limiting examples of saturated alkyl groups include methyl, ethyl, n-propyl, i-propyl, n-butyl, t-butyl, i-butyl, s-butyl, homologs and isomers of, for example, n-pentyl, n-hexyl, n-heptyl, n-octyl, and the like. Examples of lower alkyl groups include methyl, ethyl, n-propyl, i-propyl, n-butyl, s-butyl, i-butyl, and t-butyl. A substituted alkyl group is one having at least one but no more than five substituents, and no more substituents than the number of hydrogen atoms in the unsubstituted group. In some embodiments, the substituents are fluorine atoms. Non-limiting examples of substituted alkyl groups include 2-hydroxyethyl, 2-methoxyethyl, CHF₂, CF₃, CH₂CF₃, CF₂CF₃, 4-fluorobutyl, and the like.

As used herein and unless otherwise specified, the term “alkynyl” refers to a substituted or unsubstituted, linear or branched, univalent hydrocarbon chain having at least two carbon atoms and at least one carbon-carbon triple bond. Non-limiting examples of alkynyl groups include ethynyl, 1- and 3-propynyl, 3-butyn-1-yl, and the like.

As used herein and unless otherwise specified, the term “aryl”, used alone or as part of a larger moiety (for example, “(aryl)alkyl”) refers to a univalent monocyclic or bicyclic carbocyclic aromatic ring system. Unless otherwise specified, aryl groups contain 6 or 10 ring members. Non-limiting examples of aryl include phenyl, naphthyl, and the like. The term “aryl” also refers to aryl groups that may be unsubstituted or substituted. For example, aryl groups can be unsubstituted or can be substituted with one, two, or three groups selected independently from the group consisting of halogen, OH, C₁-C₆ alkoxy, substituted C₁-C₆ alkoxy, C₁-C₆ alkylthio, substituted C₁-C₆ alkylthio, C₁-C₆ alkyl, substituted C₁-C₆ alkyl, C₃-C₆ cycloalkyl, substituted C₃-C₆ cycloalkyl, C(O)OH, C(O)(C₁-C₆ alkyl), C(N—OH) (C₁-C₆ alkyl), C(O)(C₁-C₆ alkoxy), C(O)NH₂, C(O)NH(C₁-C₆ alkyl), C(O)N(C₁-C₄ alkyl) (C₁-C₄ alkyl), C(O)-heterocyclyl, NHC(O)(C₁-C₆ alkyl), N(CH₃) C(O)(C₁-C₆ alkyl), and cyano.

As used herein and unless otherwise specified, the term “azepinoindole” refers to a compound of Formula I per this disclosure.

As used herein and unless otherwise specified or clear from context, substituent “c” refers to either one of substituent “c¹” or “c²”.

As used herein and unless otherwise specified or clear from context, the term “chemical entity” refers to a compound having the indicated structure, whether in its “free” form (e.g., “free compound” or “free base” or “free acid” form, as applicable), or in a salt form, particularly a pharmaceutically acceptable salt form, and furthermore whether in solid state form or otherwise. In some embodiments, a solid state form is an amorphous (i.e., non-crystalline) form; in some embodiments, a solid state form is a crystalline form (e.g., a polymorph, pseudohydrate, hydrate, or solvate). Similarly, the term encompasses the compound whether provided in solid form or otherwise. Unless otherwise specified, all statements made herein regarding “compounds” apply to the associated chemical entities, as defined.

As used herein and unless otherwise specified, the terms “comprising”, “having”, “including”, “containing”, and grammatical variations thereof, are inclusive or open-ended and do not exclude additional, un-recited elements and/or method steps. For example, “A includes 1, 2, and 3” means that A includes but is not limited to 1, 2, and 3.

As used herein and unless otherwise specified, the term “consisting essentially of” when used herein in connection with a composition, use, or method, denotes that additional elements, method steps or both additional elements and method steps may be present, but that these additions do not materially affect the manner in which the recited composition, method, or use functions.

As used herein and unless otherwise specified, the term “consisting of” when used herein in connection with a composition, use, or method, excludes the presence of additional elements and/or method steps.

As used herein and unless otherwise specified, the term “cycloalkyl”, used alone or as part of a larger moiety, for example “(cycloalkyl)alkyl”, refers to: (i) a substituted or unsubstituted, univalent monocyclic hydrocarbon radical that is completely saturated or that contains one or more units of unsaturation, but which is not aromatic; or (ii) bicyclo[m.n.o] alkyl wherein each of “m”, “n”, and “o” is independently an integer ranging from zero to 5, and the sum “m″+” n″+ “0” ranges from 2 to 6. In some embodiments, cycloalkyl groups contain 3 to 8 ring carbon atoms (“C₃-C₈ cycloalkyl”). Non-limiting examples of cycloalkyl include cyclopropyl, cyclobutyl, cyclopentyl, cyclohexyl, 1-cyclohexenyl, 3-cyclohexenyl, cycloheptyl, and the like, as well as bicyclo[2.2.1]heptyl (also called norbornyl) and bicyclo[1.1.1]pentyl. A substituted cycloalkyl group is one having at least one but no more than five substituents. In some embodiments, the substituents are fluorine atoms. Non-limiting examples of substituted cycloalkyl groups include 2-methylcyclopropyl, 4-hydroxycyclohexyl, 2-methoxycyclopentyl, 4,4-difluorocyclohexyl, and the like.

As used herein and unless otherwise specified or clear from context, substituent “d” refers to either one of substituent “d¹” or “d²”.

As used herein and unless otherwise specified or clear from context, substituent “e” refers to either one of substituent “e¹” or “e^{2”}.

As used herein and unless otherwise specified or clear from context, substituent “f” refers to either one of substituent “f¹” or “f²”.

As used herein and unless otherwise specified, the term “halogen” or “halo”, used alone or as part of a larger moiety, refers to fluoro, chloro, bromo, or iodo.

As used herein and unless otherwise specified, the term “heteroalkyl” refers to a substituted or unsubstituted, saturated or unsaturated alkyl group, as defined herein, in which one or more of the constituent carbon atoms have been replaced by nitrogen, oxygen, or sulfur.

As used herein and unless otherwise specified, the term “heteroaryl”, used alone or as part of a larger moiety, e.g., “(heteroaryl)alkyl”, refers to a univalent monocyclic or bicyclic group having 5 to 10 ring atoms, preferably 5, 6, 9, or 10 ring atoms, having 6 or 10 TT electrons shared in a cyclic array, and having, in addition to ring carbon atoms, from one to four ring heteroatoms. Examples of heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolyl, indolizinyl, benzofuranyl, benzothiophenyl, benzimidazolyl, benzoxazolyl, benzothiazolyl, benzotriazolyl, quinolyl, isoquinolyl, purinyl, naphthyridinyl, pteridinyl, and the like. Heteroaryl groups may be unsubstituted or may be substituted with one, two, or three groups selected independently from halogen, OH, C₁-C₆ alkoxy, substituted C₁-C₆ alkoxy, C₁-C₆ alkylthio, substituted C₁-C₆ alkylthio, C₁-C₆ alkyl, substituted C₁-C₆ alkyl, C₃-C₆ cycloalkyl, substituted C₃-C₆ cycloalkyl, C(O)OH, C(O)(C₁-C₆ alkoxy), C(O)NH₂, C(O)NH(C₁-C₆ alkyl), C(O)N(C₁-C₄ alkyl) (C₁-C₄ alkyl), C(O)-heterocyclyl, NHC(O)(C₁-C₆ alkyl), N(CH₃) C(O)(C₁-C₆ alkyl), and cyano.

As used herein and unless otherwise specified, the term “heterocyclyl”, used alone or as part of a larger moiety (for example, “(heterocyclyl)alkyl”) refers to a univalent stable 4- to 7-membered monocyclic or 7- to 10-membered bicyclic heterocyclic moiety that is either saturated or partially unsaturated, and has, in addition to ring carbon atoms, one to four heteroatoms. Non-limiting examples of heterocyclyl groups include tetrahydrofuranyl, pyrrolidinyl, tetrahydropyranyl, piperidinyl, morpholinyl, and the like. Heterocyclyl groups can be unsubstituted or can be substituted. For example, heterocyclyl groups can be unsubstituted or can be substituted with one, two, or three groups selected independently from the group consisting of halogen, OH, O(C₁-C₆ alkyl), O (substituted C₁-C₆ alkyl), C₁-C₆ alkyl, substituted C₁-C₆ alkyl, and C₃-C₆ cycloalkyl.

As used herein and unless otherwise specified, the term “inactive” (and all related terms thereto including “inactivity”), when used the context of “EC₅₀ (nM)” and “Eff %” as such terms would be understood by a person skilled in the art or equivalent skilled person, and when used in reference to the activity at the 5-HT_{2B} receptor, means a concentration of greater than 10,000 nM (when used in the context of “EC₅₀ (nM)”) or an efficacy of 30% or lower (when used in the context of “Eff %”).

As used herein and unless otherwise specified, the term “isotopologue” refers to a species that differs from a specific compound only in the isotopic composition thereof. For example, all hydrogen atoms in a compound are independently of natural isotopic composition or of any isotopic composition enriched or depleted in one or both of the heavy isotopes, 2H (D, deuterium) and 3H (T, tritium), ranging from a depletion to zero % to an enrichment to 100%.

As used herein and unless otherwise specified, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts of the compounds provided in this disclosure include salts derived from suitable inorganic and organic acids and bases. Non-limiting examples of pharmaceutically acceptable salts include salts of compounds comprising an amino group that are formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid. Other non-limiting examples of pharmaceutically acceptable salts include adipate, alginate, ascorbate, aspartate, benzenesulfonate, benzoate, bisulfate, borate, butyrate, camphorate, camphorsulfonate, cyclopentanepropionate, digluconate, dodecylsulfate, ethanesulfonate, formate, fumarate, glucoheptonate, glycerophosphate, gluconate, hemisulfate, heptanoate, hexanoate, hydriodide, 2-hydroxyethanesulfonate, lactobionate, laurate, lactate, lauryl sulfate, malate, methanesulfonate, 2-naphthalenesulfonate, nicotinate, nitrate, oleate, palmitate, pamoate, pectinate, persulfate, 3-phenylpropionate, pivalate, propionate, stearate, thiocyanate, p-toluenesulfonate, undecanoate, valerate salts, and the like. Other pharmaceutically acceptable salts include those that are derived from appropriate bases such as alkali metal, alkaline earth metal, ammonium, and N+ (C₁-4 alkyl) 4 salts. Representative alkali or alkaline earth metal salts include sodium, lithium, potassium, calcium, magnesium, and the like. Further non-limiting examples of pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein and unless otherwise specified, the term “subject” includes a mammal (e.g., a human, and in some embodiments including prenatal human forms). In some embodiments, a subject suffers from a relevant disease, disorder, or condition. In some embodiments, a subject is susceptible to a disease, disorder, or condition. In some embodiments, a subject displays one or more symptoms or characteristics of a disease, disorder, or condition. In some embodiments, a subject does not display any symptom or characteristic of a disease, disorder, or condition. In some embodiments, a subject is a mammal with one or more features characteristic of susceptibility to or risk of a disease, disorder, or condition. In some embodiments, a subject is a patient. In some embodiments, a subject is an individual to whom diagnosis and/or therapy is and/or has been administered. In some embodiments, a subject is a fetus, an infant, a child, a teenager, an adult, or a senior citizen (i.e., the subject is of advanced age, such as older than 50). In some embodiments, a child refers to a human that is between two and 18 years of age. In some embodiments, an adult refers to a human that is eighteen years of age or older.

As used herein and unless otherwise specified, the phrase “such as” is intended to be open-ended. For example, the phrase “A can be a halogen, such as chlorine or bromine” means that “A” can be, but is not limited to, chlorine or bromine.

Reference to specific moieties, functional groups, or substituents contemplates (where applicable) tautomers thereof.

Unless otherwise stated, structures depicted herein include all isomeric (e.g., enantiomeric, diastereomeric, and geometric (or conformational)) forms of the structure (e.g., the R and S configurations for each asymmetric center, Z and E double bond isomers, and Z and E conformational isomers). Unless otherwise stated, the compounds disclosed, taught, or otherwise suggested in this disclosure contemplate all single stereochemical isomers as well as enantiomeric, diastereomeric, and geometric (or conformational) mixtures thereof. Unless otherwise stated, the compounds disclosed, taught, or suggested in this disclosure contemplate all tautomeric forms thereof. Additionally, unless otherwise stated, structures depicted herein include compounds that differ only in the presence of one or more isotopically enriched atoms. Such compounds may be useful, for example, as analytical tools, as probes in biological assays, or as therapeutic agents. Additionally, incorporation of heavier isotopes such as deuterium (2H) may afford certain therapeutic advantages resulting from greater metabolic stability, for example, increase in vivo half-life, or reduced dosage requirements.

Chemical entities described herein are further illustrated by the classes, subclasses, and species disclosed herein. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. In this disclosure, any atom not specifically designated as a particular isotope is meant to represent any stable isotope of that atom.

Unless otherwise stated, structures depicted herein are also meant to include all stereoisomeric (e.g., enantiomeric, diastereomeric, and geometric (or conformational)) forms of the structure; for example, the R and S configurations for each asymmetric center, Z and E double bond isomers, and Z and E conformational isomers. Therefore, the present compounds contemplate all single stereochemical isomers as well as enantiomeric, diastereomeric, and geometric (or conformational) mixtures thereof. Unless otherwise stated, the present compounds contemplate all tautomeric forms thereof. Additionally, unless otherwise stated, structures depicted herein are also meant to include compounds that differ only in the presence of one or more isotopically enriched atoms. Such compounds may be useful, for example, as analytical tools, as probes in biological assays, or as therapeutic agents. Additionally, incorporation of heavier isotopes such as deuterium (2H) may afford certain therapeutic advantages resulting from greater metabolic stability, for example, increase in vivo half-life, or reduced dosage requirements.

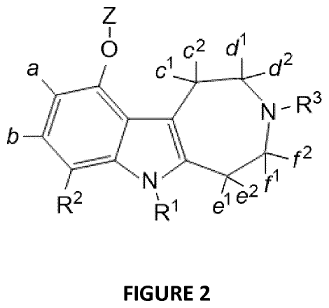

Referring to FIG. 2, and according to some embodiments of the chemical entities disclosed herein (including isotopologues or pharmaceutically acceptable salts thereof), there are chemical entities of Formula I:

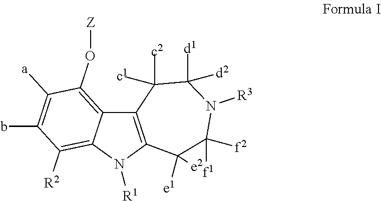

R¹: (i) is selected from the group consisting of H, C₁-C₆ alkyl, C₁-C₆ substituted alkyl, C₂-C₆ alkenyl, C₂-C₆ alkynyl, C₃-C₆ cycloalkyl, (C₃-C₆ cycloalkyl) (C₁-C₆ alkyl), C₃-C₆ heterocyclyl, (C₃-C₆ heterocyclyl) (C₁-C₆ alkyl), aryl(C₁-C₆ alkyl), and heteroaryl(C₁-C₆ alkyl); or (ii) together with e¹ or e² form a chain of 2 to 4 carbon atoms to which are attached substituents independently selected from the group consisting of H, C₁-C₆ alkyl, aryl, heteroaryl, and any combination thereof.

R²: (i) is selected from the group consisting of C₁-C₆ alkyl, C₁-C₆ substituted alkyl, C₂-C₆ alkenyl, C₂-C₆ alkynyl, C₃-C₆ cycloalkyl, (C₃-C₆ cycloalkyl) (C₁-C₆ alkyl), C₃-C₆ heterocyclyl, (C₃-C₆ heterocyclyl) (C₁-C₆ alkyl), aryl, aryl(C₁-C₆ alkyl), heteroaryl, heteroaryl(C₁-C₆ alkyl), CN, C(O)NH₂, C(O)NH(C₁-C₆ alkyl), C(O)N(C₁-C₃ alkyl) (C₁-C₆ alkyl), C(═NOH) (C₁-C₆ alkyl), and C(═NOH) (C₁-C₆ substituted alkyl), phenyl, and halogen; or (ii) together with b form a chain of 2 or 3 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCF₃, cyano, and oxo; or (iv) is selected from the group consisting of H, C₁-C₆ alkyl, C₁-C₆ substituted alkyl, C₂-C₆ alkenyl, C₂-C₆ alkynyl, C₃-C₆ cycloalkyl, (C₃-C₆ cycloalkyl) (C₁-C₆ alkyl), C₃-C₆ heterocyclyl, (C₃-C₆ heterocyclyl) (C₁-C₆ alkyl), aryl, aryl(C₁-C₆ alkyl), heteroaryl, heteroaryl(C₁-C₆ alkyl), CN, C(O)NH₂, C(O)NH(C₁-C₆ alkyl), C(O)N(C₁-C₃ alkyl) (C₁-C₆ alkyl), C(═NOH) (C₁-C₆ alkyl), and C(═NOH) (C₁-C₆ substituted alkyl), if b is halogen, CH₃, CHF₂, CF₃, OCH₃, OCHF₂, OCF₃, SCH₃, SCHF₂, SCF₃, or cyano. In some embodiments, R² together with b form any one of CH₂CH₂, CH₂CH₂CH₂, CH₂CH₂CH₂CH₂, CH═CHCH═CH, OCH₂CH₂, CH₂OCH₂, CH₂CH₂O, OCH═CH, CH═CHO, OCH₂O, SCH₂CH₂, CH₂SCH₂, CH₂CH₂S, SCH═CH, CH═CHS, NHCH₂CH₂, CH₂NHCH₂, CH₂CH₂NH, NHCH═CH, CH═CHNH, ON═CH, CH═NO, OCH═N, N═CHO, SN═CH, CH═NS, SCH═N, N═CHS, NHN═CH, CH═NNH, NHCH═N, N═CHNH, NHN═N, N═NNH, OCH₂CH₂CH₂, CH₂OCH₂CH₂, CH₂CH₂OCH₂, CH₂CH₂CH₂O, SCH₂CH₂CH₂, CH₂SCH₂CH₂, CH₂CH₂SCH₂, CH₂CH₂CH₂SNHCH₂CH₂CH₂, CH₂NHCH₂CH₂, CH₂CH₂NCH₂, CH₂CH₂CH₂NH, N═CHCH═CH, CH═NCH═CH, CH═CHN═CH, CH═CHCH═N. In some embodiments, R² together with b form any one of CH₂CH₂, CH₂CH₂CH₂, CH₂CH₂CH₂CH₂, CH═CHCH═CH, OCH₂CH₂, CH₂OCH₂, CH₂CH₂O, OCH═CH, CH═CHO, OCH₂O, SCH₂CH₂, CH₂SCH₂, CH₂CH₂S, SCH═CH, CH═CHS, NHCH₂CH₂, CH₂NHCH₂, CH₂CH₂NH, NHCH═CH, CH═CHNH, ON═CH, CH═NO, OCH═N, N═CHO, SN═CH, CH═NS, SCH═N, N═CHS, NHN═CH, CH═NNH, NHCH═N, N═CHNH, NHN═N, N═NNH, OCH₂CH₂CH₂, CH₂OCH₂CH₂, CH₂CH₂OCH₂, CH₂CH₂CH₂O, SCH₂CH₂CH₂, CH₂SCH₂CH₂, CH₂CH₂SCH₂, CH₂CH₂CH₂S NHCH₂CH₂CH₂, CH₂NHCH₂CH₂, CH₂CH₂NCH₂, CH₂CH₂CH₂NH, N═CHCH═CH, CH═NCH═CH, CH═CHN═CH, CH═CHCH═N, wherein one hydrogen atom or two hydrogen atoms, if present on a moiety, are replaced with substituents selected independently from the group consisting of halogen, OH, C₁-C₆ alkoxy, C₁-C₆ alkyl, C₃-C₆ cycloalkyl, CHF₂, CF₃, OCHF₂, OCF₃, SCH₃, SCF₃, and cyano, or wherein two hydrogens, if attached to the same carbon atom, are replaced with an oxo group.

a: (i) is selected from the group consisting of H, halogen, lower alkyl, CHF₂, CF₃, OCH₃, OCHF₂, OCF₃, SCHF₂, SCH₃, SCF₃, amine, and cyano; or (ii) together with Z form one of (A) a saturated chain of one oxygen and one carbon atom (with oxygen connected to the 5-position of the indole ring of Formula I), and (B) a chain of 2 or 3 carbon atoms, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo, and (C) a chain of 2 or 3 carbon atoms containing one double bond, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCHF₂, SCH₃, SCF₃, cyano, and oxo; or (iii) together with b form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo.

b: (i) is selected from a group consisting of H, halogen, CH₃, CHF₂, CF₃, OCH₃, OCHF₂, OCF₃, SCH₃, SCHF₂, SCF₃, amine and cyano; or (ii) together with a form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo; or (iii) together with R² form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo.

R³: (i) is selected from the group consisting of H, C₁-C₆ alkyl, C₂-C₆ alkenyl, C₂-C₆ alkynyl, C₃-C₆ cycloalkyl, (C₃-C₆ cycloalkyl) (C₁-C₆ alkyl), aryl(C₁-C₆ alkyl), acetyl, and heteroaryl(C₁-C₆ alkyl); or (ii) together with f and the N atom to which R³ is attached form an azetidine or pyrrolidine ring, such ring carrying substituents independently selected from the group consisting of H, aryl, heteroaryl, C₁-C₆ alkyl, and C₃-C₆ cycloalkyl; or (iii) together with d and the N atom to which R³ is attached form an azetidine or pyrrolidine ring, such ring carrying substituents independently selected from the group consisting of H, aryl, heteroaryl, halogen, C₁-C₆ alkyl, and C₃-C₆ cycloalkyl.

Each of c, d, e, and f is H or a lower alkyl group; or c¹ and c² together form a part of a spiro-fused cyclopropane or cyclobutane ring, while each of d, e, and fare H or lower alkyl groups; or d¹ and d² together form a part of a spiro-fused cyclopropane or cyclobutane ring, while each of c, e, and f are H or lower alkyl groups; or e¹ and e² together form a part of a spiro-fused cyclopropane or cyclobutane ring, while each of c, d, and fare H or lower alkyl groups; or f¹ and f² together form a part of a spiro-fused cyclopropane or cyclobutane ring, while each of c, d, and e are H or lower alkyl groups; or one of c and one of d together form —CH₂— or —CH₂CH₂—, thereby giving rise to a condensed cyclopropane or cyclobutane ring, while the remainder of each of c, d, e, and f are H or lower alkyl groups; or one of e and one of f together form —CH₂— or —CH₂CH₂—, thereby giving rise to a condensed cyclopropane or cyclobutane ring, while the remainder of each of c, d, e, and f are H or lower alkyl groups; or one of c and one of e together form —CH₂— or —CH₂CH₂—, thereby giving rise to a bridged bicyclic substructure, while the remainder of each of c, d, e, and f are H or lower alkyl groups; or one of c and one of f together form —CH₂— or —CH₂CH₂—, thereby giving rise to a bridged bicyclic substructure, while the remainder of each of c, d, e, and f are H or lower alkyl groups; or one of d and one of e together form —CH₂— or —CH₂CH₂—, thereby giving rise to a bridged bicyclic substructure, while the remainder of each of c, d, e, and f are H or lower alkyl groups; or one of f and one of e together form —CH₂— or —CH₂CH₂—, thereby giving rise to a condensed bicyclic substructure, while the remainder of each of c, d, e, and f are H or lower alkyl groups; or e together with R¹ form a chain of 2 to 4 carbon atoms to which are attached substituents independently selected from the group consisting of H, C₁-C₆ alkyl, aryl, heteroaryl, and any combination thereof; or one of c and R³ together form —CH₂— or —CH₂CH₂—, thereby giving rise to a bridged bicyclic substructure, while the remainder of each of c, d, e, and fare H or lower alkyl groups; or one of d and R³ together form —CH₂— or —CH₂CH₂—, thereby giving rise to a condensed bicyclic substructure, while the remainder of each of c, d, e, and fare H or lower alkyl groups; or one of e and R³ together form —CH₂— or —CH₂CH₂—, thereby giving rise to a condensed bicyclic substructure, while the remainder of each of c, d, e, and fare H or lower alkyl groups; or one of f and R³ together form —CH₂— or —CH₂CH₂—, thereby giving rise to a bridged bicyclic substructure, while the remainder of each of c, d, e, and fare H or lower alkyl groups.

Z: (i) is selected from the group consisting of H, R⁵, (R⁶) (R⁷)N—C(O)—, C₁-C₆ alkyl-C(O), C₃-C₆ cycloalkyl-C(O), aryl-C(O), and heteroaryl-C(O), wherein R⁵ is selected from the group consisting of C₁-C₆ alkyl, C₂-C₆ alkenyl, C₂-C₆ alkynyl, C₃-C₆ cycloalkyl, (C₃-C₆ cycloalkyl) (C₁-C₆ alkyl), aryl(C₁-C₆ alkyl), and heteroaryl(C₁-C₆ alkyl), and wherein R⁶ and R⁷ are each independently selected from the group consisting of H, C₁-C₄ alkyl, and C₃-C₆ cycloalkyl or are joined to form a 4-7 membered heterocyclyl group; or (ii) is (R⁸O) (R⁹O) P(O)—, wherein R⁸ and R⁹ are each independently H or a cationic counterion of a phosphate salt form such as sodium, potassium, one-half of magnesium, one-half of calcium, ammonium, or ammonium substituted with one or more alkyl or cycloalkyl groups; or (iii) together with c form a linkage that gives rise to a pyran or oxepan ring comprising substituents independently selected from the group consisting of H, halogen, C₁-C₆ alkyl, and C₃-C₆ cycloalkyl; or (iv) together with a form one of (A) a saturated chain of one oxygen and one carbon atom (with oxygen connected to the 5-position of the indole ring of Formula I), and (B) a chain of 2 or 3 carbon atoms, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo, and (C) a chain of 2 or 3 carbon atoms containing one double bond and carrying substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo.

### Examples of Embodiments of Chemical Entities

Examples of chemical entities of Formula I are shown in Table 1 below.

TABLE 1
Examples of Compounds of Formula I *

| | 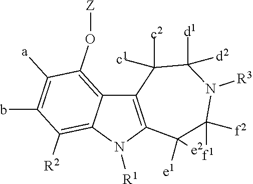 | | | | | | | | | | | | | |
|---|---|---|---|---|---|---|---|---|---|---|---|---|---|---|
| | Formula I | | | | | | | | | | | | | |
| cpd# | Z | a | b | R² | R¹ | e¹ | e² | f¹ | f² | R³ | d¹ | d² | c¹ | c² |
| 1 (136) | H | H | H | Me | H | H | H | H | H | H | H | H | H | H |
| 2 | Me | H | H | Me | H | H | H | H | H | H | H | H | H | H |
| 3 | i-Pr | H | H | Me | H | H | H | H | H | H | H | H | H | H |
| 4 | Ph | H | H | Me | H | H | H | H | H | H | H | H | H | H |
| 5 | Bn | H | H | Me | H | H | H | H | H | H | H | H | H | H |
| 6 | Piv | H | H | Me | H | H | H | H | H | H | H | H | H | H |
| 7 | Z¹ | H | H | Me | H | H | H | H | H | H | H | H | H | H |
| 8 | Z² | H | H | Me | H | H | H | H | H | H | H | H | H | H |
| 9 | Z³ | H | H | Me | H | H | H | H | H | H | H | H | H | H |
| 10 | Z⁴ | H | H | Me | H | H | H | H | H | H | H | H | H | H |
| 11 | Z⁵ | H | H | Me | H | H | H | H | H | H | H | H | H | H |
| 12 | H | H | H | Me | Me | H | H | H | H | H | H | H | H | H |
| 13 | H | H | H | Me | Bn | H | H | H | H | H | H | H | H | H |
| 14 (W5) | H | H | H | Me | H | H | H | H | H | Me | H | H | H | H |
| 15 | H | H | H | Me | H | H | H | H | H | Et | H | H | H | H |
| 16 | H | H | H | Me | H | H | H | H | H | CyprCH₂ | H | H | H | H |
| 17 | H | H | H | Me | H | H | H | TH | H | Bn | H | H | IH | H |
| 18 (A90) | H | H | H | Me | H | H | H | H | H | HOBn | H | H | H | H |
| 19 | H | H | H | Me | H | H | H | H | H | MeOBn | H | H | H | H |
| 20 | H | H | H | Me | H | H | H | H | H | MeSBn | H | H | H | H |
| 21 | H | H | H | Et | H | H | H | H | H | H | H | H | H | H |
| 22 | H | H | H | n-Pr | H | H | H | H | H | H | H | H | H | H |
| 23 | H | H | H | i-Pr | H | H | H | H | H | H | H | H | H | H |
| 24 | H | H | H | Cypr | H | H | H | H | H | H | H | H | H | H |
| 25 | H | H | H | Ph | H | H | H | H | H | H | H | H | H | H |
| 26 | H | H | H | Bn | H | H | H | H | H | H | H | H | H | H |
| 27 (A92) | H | F | H | Me | H | H | H | H | H | H | H | H | H | H |
| 28 | H | Cl | H | Me | H | H | H | H | H | H | H | H | H | H |
| 29 | H | Br | H | Me | H | H | H | H | H | H | H | H | H | H |
| 30 | H | CN | H | Me | H | H | H | H | H | H | H | H | H | H |
| 31 | H | Me | H | Me | H | H | H | H | H | H | H | H | H | H |
| 32 | H | H | F | Me | H | H | H | H | H | H | H | H | H | H |
| 33 | H | H | Cl | Me | H | H | H | H | H | H | H | H | H | H |
| 34 | H | H | Br | Me | H | H | H | H | H | H | H | H | H | H |
| 35 | H | H | CN | Me | H | H | H | H | H | H | H | H | H | H |
| 36 | H | H | Me | Me | H | H | H | H | H | H | H | H | H | H |
| 37 | H | H | (CH₂)₃ | | H | H | H | H | H | H | H | H | H | H |
| 38 | H | H | OCH₂CH₂ | | H | H | H | H | H | H | H | H | H | H |
| 39 | H | H | OCH═CH | | H | H | H | H | H | H | H | H | H | H |
| 40 | H | H | CH₂OCH₂ | | H | H | H | H | H | H | H | H | H | H |
| 41 | CH₂CH₂ | | H | Me | H | H | H | H | H | H | H | H | H | H |
| 42 | (CH₂)₃ | | H | Me | H | H | H | H | H | H | H | H | H | H |
| 43 | CH═ | | H | Me | H | H | H | H | H | H | H | H | H | H |
| | CH | | | | | | | | | | | | | |
| 44 | CH₂O | | H | Me | H | H | H | H | H | H | H | H | H | H |
| 45 (W8) | H | H | H | Me | H | Me | H | H | H | H | H | H | H | H |
| 46 | H | H | H | Me | H | Me | Me | H | H | H | H | H | H | H |
| 47 | H | H | H | Me | H | CH₂CH₂ | | H | H | H | H | H | H | H |
| 48 | H | H | H | Me | H | H | H | Me | H | H | H | H | H | H |
| 49 | H | H | H | Me | H | H | H | Me | Me | H | H | H | H | H |
| 50 | H | H | H | Me | H | H | H | CH₂CH₂ | | H | H | H | H | H |
| 51 | H | H | H | Me | H | H | H | H | H | H | Me | H | H | H |
| 52 | H | H | H | Me | H | H | H | H | H | H | Me | Me | H | H |
| 53 | H | H | H | Me | H | H | H | H | H | H | CH₂CH₂ | | H | H |
| 54 | H | H | H | Me | H | H | H | H | H | H | H | H | Me | H |
| 55 | H | H | H | Me | H | H | H | H | H | H | H | H | Me | Me |
| 56 | H | H | H | Me | H | H | H | H | H | H | H | H | CH₂CH₂ | |
| 57 | H | H | H | Me | CH₂CH₂ | | H | H | H | H | H | H | H | H |
| 58 | H | H | H | Me | CH(Ph)CH₂ | | H | H | H | H | H | H | H | H |
| 59 | H | H | H | Me | (CH₂)₃ | | H | H | H | H | H | H | H | H |
| 60 | H | H | H | Me | H | H | CH₂ | | H | H | H | H | H | H |
| 61 | H | H | H | Me | H | H | CH₂CH₂ | | H | H | H | H | H | H |
| 62 | H | H | H | Me | H | H | H | H | H | H | H | CH₂ | | H |
| 63 | H | H | H | Me | H | H | H | H | H | H | H | CH₂CH₂ | | H |
| 64 (A96) | H | H | H | H | Me | H | H | H | H | Me | H | H | H | H |
| 65 (A95) | H | H | H | Me | Me | H | H | H | H | H | H | H | H | H |
| 66 (A94) | H | H | H | H | H | H | H | H | H | Me | H | H | H | H |
| 67 (A93) | H | H | H | H | H | H | H | H | H | H | H | H | H | H |
| cpd# | Z | a | b | R² | R¹ | c¹ | f¹ | d¹ | e² | c² | f² | d² | e² | R³ |

| | | | | | | | | | | | | | |
|---|---|---|---|---|---|---|---|---|---|---|---|---|---|
| 65 | H | H | H | Me | H | CH₂ | H | H | H | H | H | H | H |
| 66 | H | H | H | Me | H | CH₂CH₂ | H | H | H | H | H | H | H |

| | | | | | | | | | | | | | |
|---|---|---|---|---|---|---|---|---|---|---|---|---|---|
| 67 | H | H | H | Me | H | H | CH₂ | H | H | H | H | H | H |
| 68 | H | H | H | Me | H | H | CH₂CH₂ | H | H | H | H | H | H |

| | | | | | | | | | | | | | |
|---|---|---|---|---|---|---|---|---|---|---|---|---|---|
| 69 (W9) | H | H | H | Me | H | H | H | CH₂ | H | H | H | H | H |
| 70 | H | H | H | Me | H | H | H | CH₂CH₂ | H | H | H | H | H |

| | | | | | | | | | | | | | |
|---|---|---|---|---|---|---|---|---|---|---|---|---|---|
| 71 (W6) | H | H | H | Me | H | H | H | H | CH₂ | H | H | H | H |
| 72 (W7) | H | H | H | Me | H | H | H | H | CH₂ | H | H | H | Me |
| 73 | H | H | H | Me | H | H | H | H | CH₂CH₂ | H | H | H | H |

*Abbreviations used in the table above have the following meanings:
Bn = benzyl;
Cypr = cyclopropyl;
Et = ethyl;
HOBn = o-hydroxybenzyl;
MeOBn—o-methoxybenzyl;
MeSBn = o-(methylthio)benzyl;
Ph = phenyl;
Piv = pivaloyl;
Pr = propyl;
Z¹ = C(O)N(H)-i-Pr;
Z² = C(O)NMe₂;
Z³ = P(O)(OH)₂;
Z⁴ = P(O)(ONa)₂;
Z⁵ = P(O)(OH)(ONa).

### Chemical Synthesis

Compounds of Formula I have been prepared by two variants of the Fischer indole synthesis, examples of which is shown below.

In a first approach, a Fischer indole synthesis involving a N-protected 4-oxoazepane and a substituted phenylhydrazine resulted in a mixture of regioisomeric azepinoindoles that was separated. Substituted phenylhydrazines are available, for example, from anilines by diazotization followed by reduction, e.g., with sodium sulfite or tin (II) salts; or from aryl halides by transition metal-catalyzed amination/amidation with hydrazine hydrate (Kurandina, D. V. et al. Tetrahedron 2014, 70, 4043-4048) or with tert-butyl carbazate (Wolter, M.; Klapars, A.; Buchwald, S. L. Org. Lett. 2001, 3, 3803-3805; Jiang, L.; Lu, X.; Zhang, H.; Jiang, Y.; Ma, D. J. Org. Chem. 2009, 74, 4542-4546), followed by removal of the tert-butoxycarbonyl group with acid. A variant of the Fischer indole synthesis has recently been disclosed in which the arylhydrazine is replaced with a 1-aryl-2-methylhydrazine (see below) to typically obtain better yields of the indole under milder reaction conditions (Schmidt. M. A. J. Org. Chem. 2022, 87, 1941-1960).

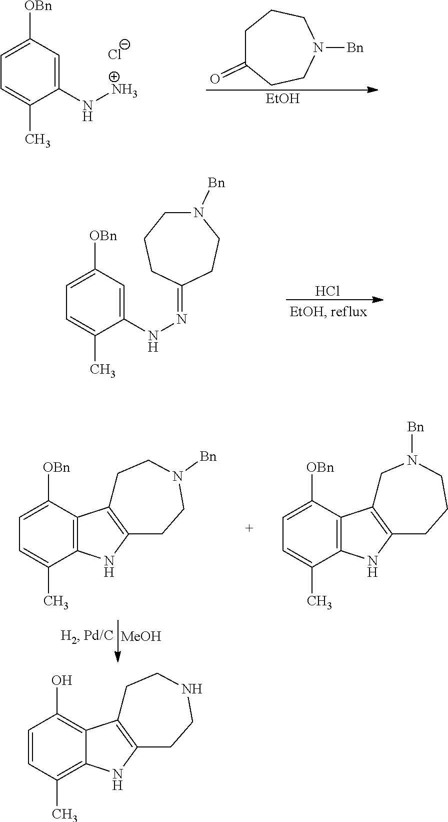

In another variant of the Fischer indole synthesis, alpha-branched aliphatic aldehydes react with arylhydrazines in the presence of acid to deliver 3,3-disubstituted 3H-indoles (indolenines) (e.g., Alfano, A. I. et al. React. Chem. Eng. 2020, 5, 2091-2100). While for the present purpose low yields were initially obtained employing the arylhydrazine in acetic acid as the solvent, replacement of this building block with the corresponding 1-aryl-2-methylhydrazine led to a more efficient reaction. The 1,2-disubstituted hydrazine starting material was available by a general literature procedure from the aryl bromide by Pd-catalyzed amination with tert-butyl 1-methylcarbazate (Mauger, C., Mignani, G. Adv. Synth. Catal. 2005, 347, 773-782; Schmidt, M. A. J. Org. Chem. 2022, 87, 1941-60).

In a subsequent step, further acid treatment of the 3,3-disubstituted 3H-indole resulted in its rearrangement to a 2,3-disubstituted 1H-indole (e. g., Rodriguez, J. G.; Temprano, F. J. Chem. Soc., Perkin Trans. 1 1988, 3243-3247; Rodriguez, J. G.; Benito, Y.; Temprano, F. J. Heterocyclic Chem. 1985, 22, 1207-1210; Wang, T. S. T. Tetrahedron Lett. 1975, 1637-1638). In the present work, the commercial, acid-washed clay mineral, montmorillonite (Kumar, B. S. Catal. Sci. Technol. 2014, 4, 2378-2396), was employed as the acid at the boiling temperature of nonpolar, high-boiling solvents such as bromobenzene or m-xylene. In a final step, both the benzyl and benzyloxycarbonyl protecting groups were removed by hydrogenolysis.

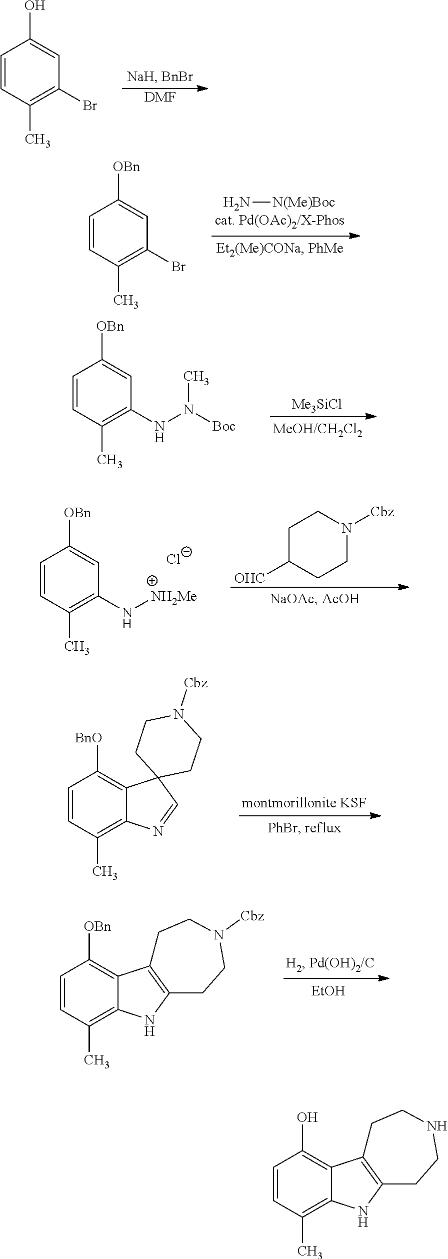

The following two schemes are presented as examples for different substituent patterns in the carbocyclic aromatic ring.

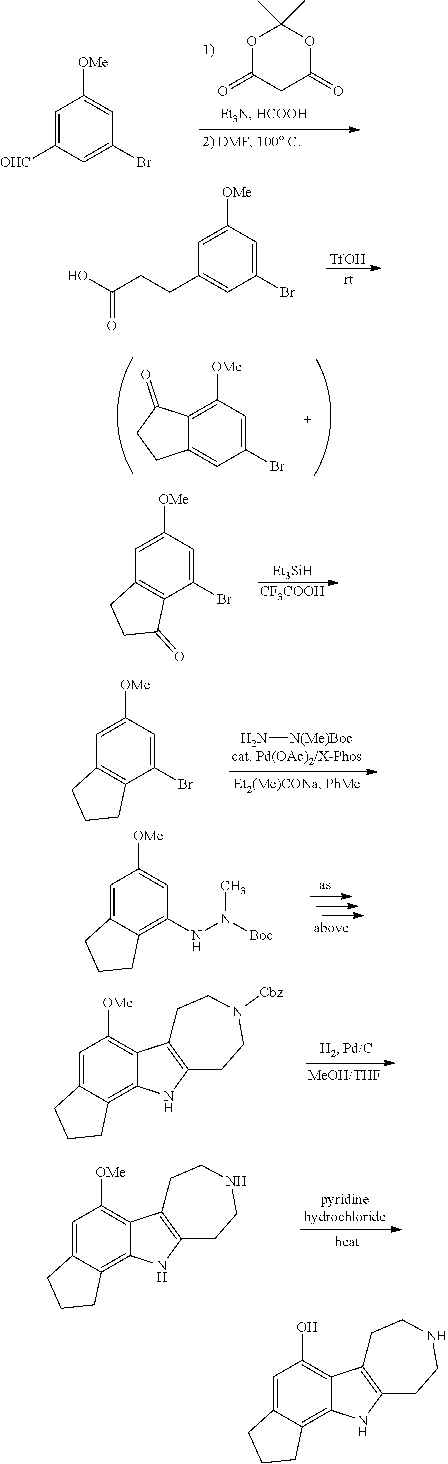

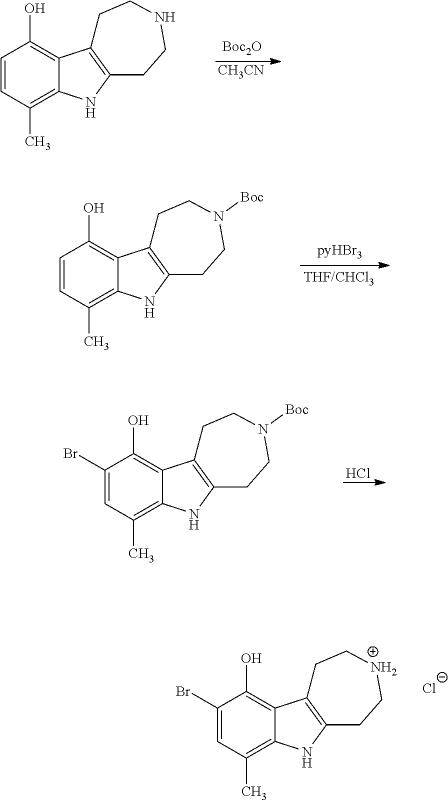

Azepanone building blocks containing a substituent e and/or a substituent f can be synthesized by photochemical rearrangement of N-alkylated succinimides (Kanaoka, Y.; Hatanaka, Y. J. Org. Chem. 1976, 41, 400-401) followed by standard functional group modifications.

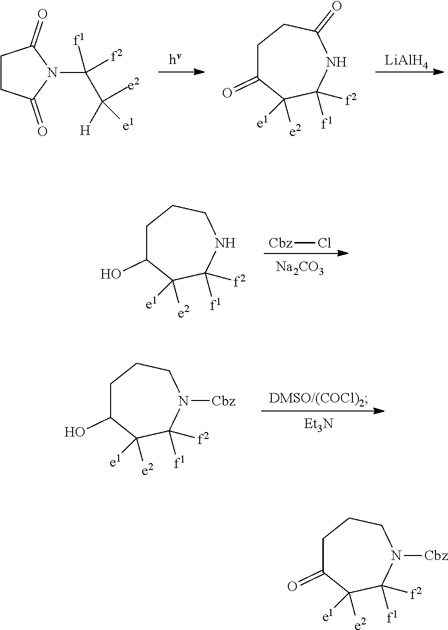

Substituted compounds of Formula I containing a substituent f can be also obtained by spirocyclization of N-sulfonylated tryptamines with tert-butyl propargyl carbonate, followed by rearrangement of the 3,3-disubstituted 3H-indole thus obtained to a 2,3-disubstituted 1H-indole (Montgomery, T. D. et al. Org. Lett. 2014, 16, 3480-3483), catalytic hydrogenation, and desulfonylation.

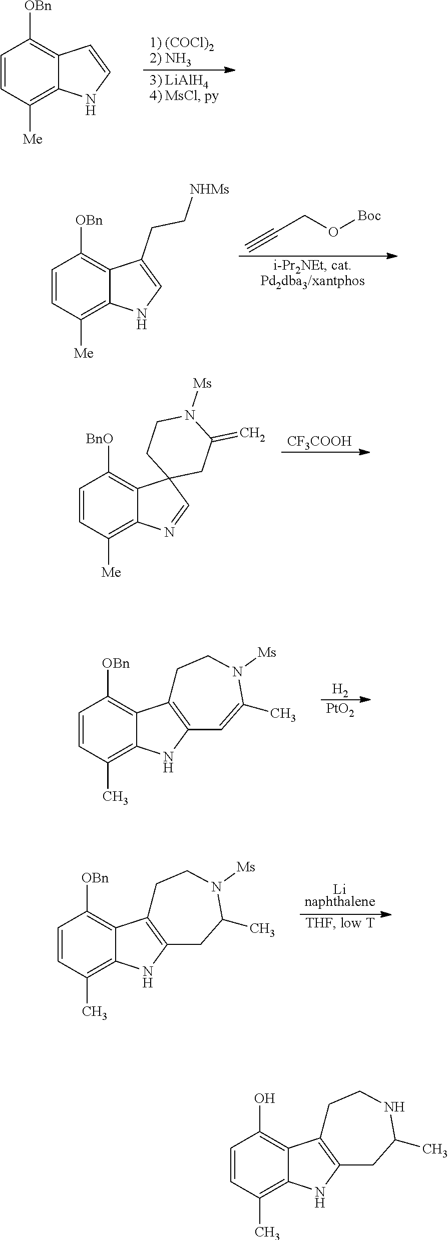

If the 2,5-azepanediones resulting from the photochemical rearrangement of N-alkylsuccinimides are entered into the Fischer indole synthesis prior to lactam reduction, the resulting indoles, after protection of both nitrogen atoms, contain an acidic methylene group adjacent to the lactam carbonyl that can be taken advantage of for the installation of one or two substituents c by deprotonation with a strong base followed by alkylation.

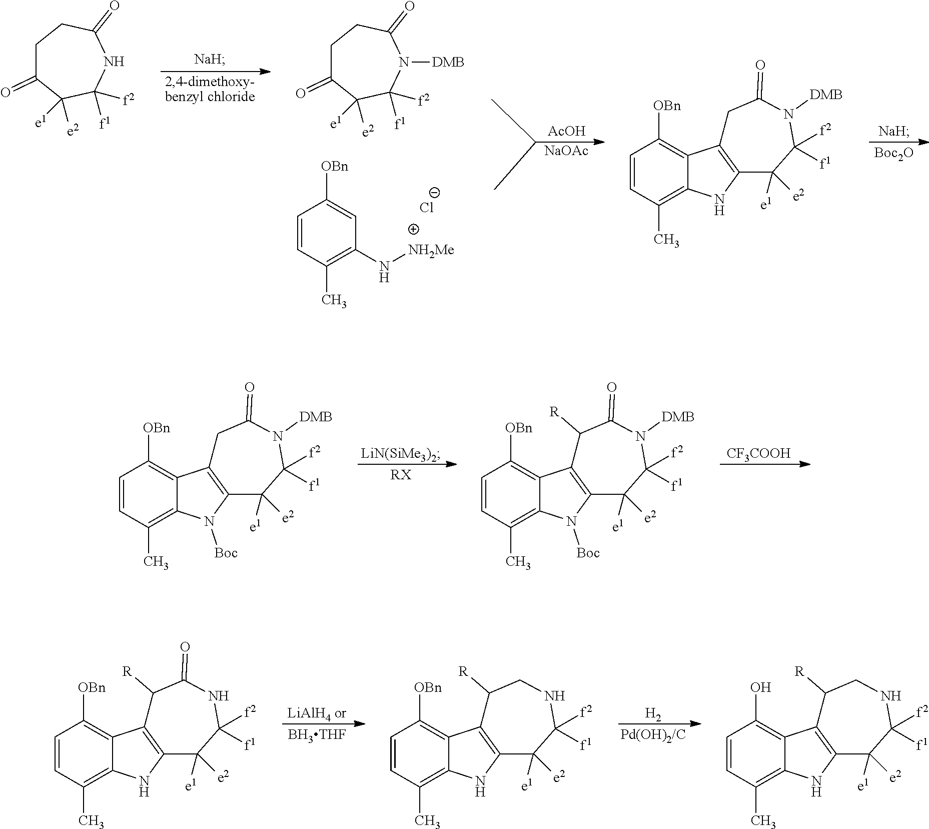

The above fully protected intermediate can also be employed to install a d substituent by reduction of the lactam function to hemiaminal, which is then nucleophilically alkylated with a Grignard reagent (e.g., Ong, D. Y. et al. Angew. Chem. Int. Ed. 2020, 59, 11903-11907).

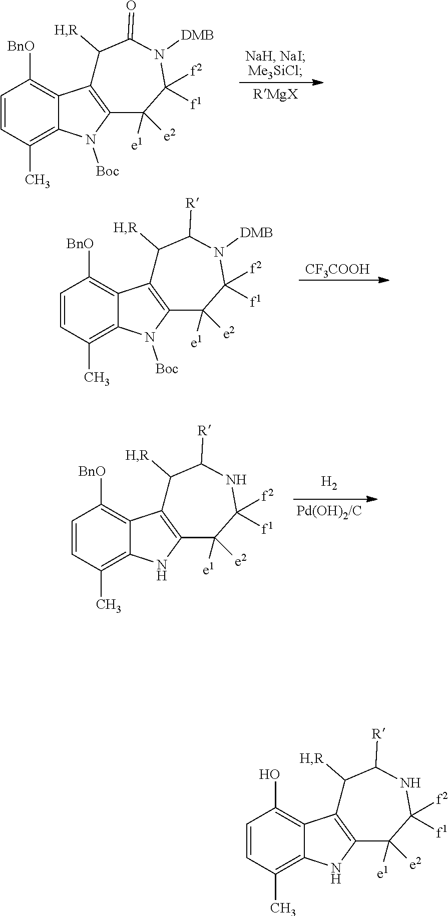

An example for the synthesis of the azabicyclic building blocks needed to obtain small-ring-annulated compounds of Formula I follows:

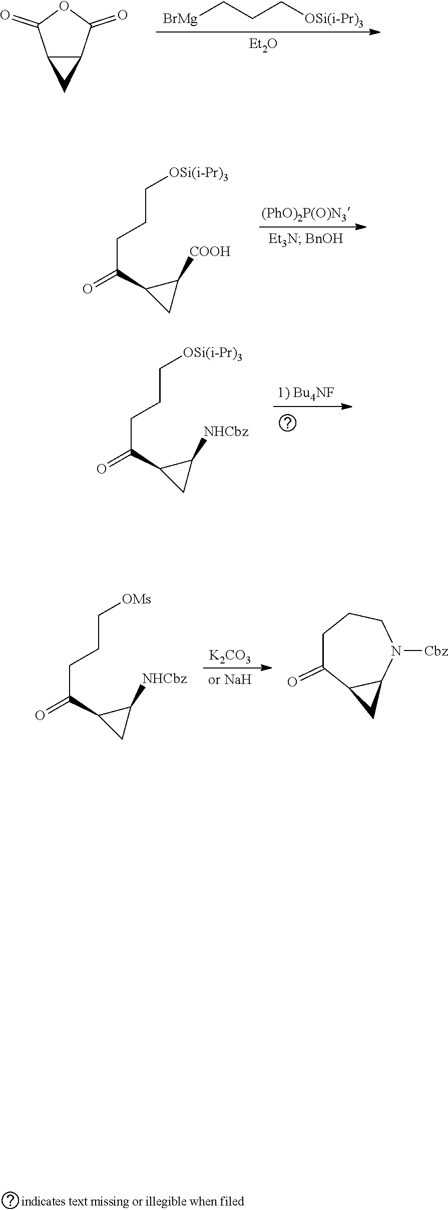

The ring-size homolog containing a four-membered ring is obtained in the same manner from the anhydride of cyclobutane-1,2-dicarboxylic acid. Additionally, carbonyl transposition (e.g., Nakai, T.; Mimura, T. Tetrahedron Lett. 1979, 531-534; review: Nakai, T.; Mimura, T. J. Synth. Org. Chem. Jpn. 1977, 35, 964-978; recent work: Wu, Z. et al. Science 2021, 374, 734-740) applied to the above ketone provides the ketone precursor for compounds of Formula I that contain a small ring in the positions carrying the substituents c and d.

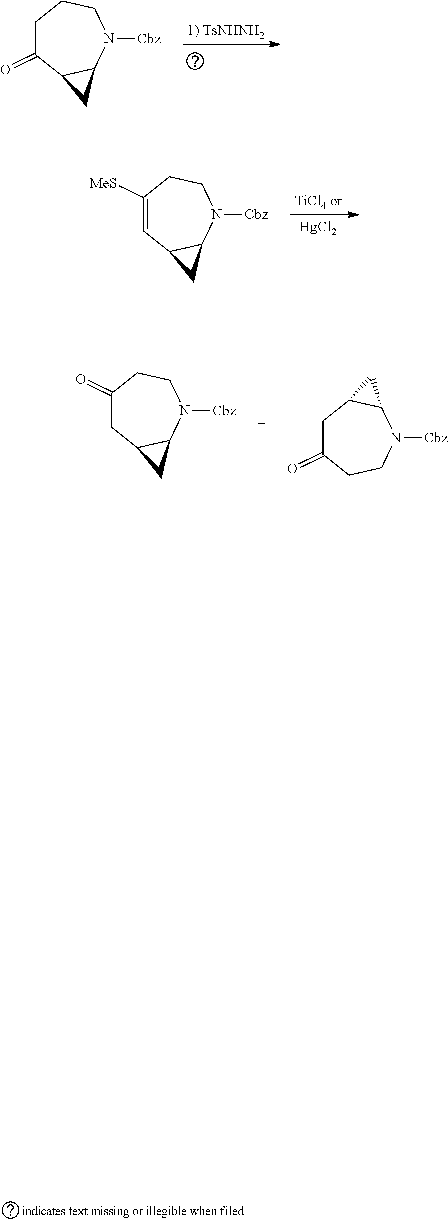

Syntheses of 3-benzyl-3-azabicyclo[3.2.1]octan-6-one and 3-benzyl-3-azabicyclo[3.2.2]nonan-6-one are disclosed in Allen, M. P. et al., US2005020830. These azabicyclic building blocks are intermediates in the synthesis of compounds of Structure I in which the substituents c and e are joined to form a bridge across the seven-membered ring.

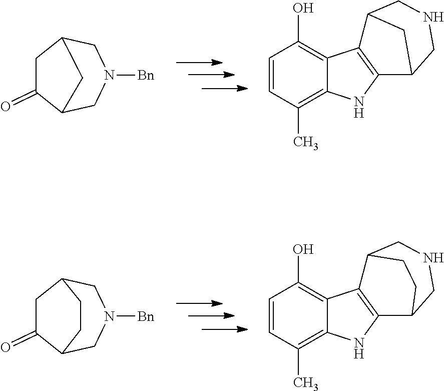 [SMILES: O=C1CC2CC1CN(Cc1ccccc1)C2 ; Cc1ccc(O)c2c3c([nH]c12)C1CNCC3C1 ; O=C1CC2CCC1CN(Cc1ccccc1)C2 ; Cc1ccc(O)c2c3c([nH]c12)C1CCC3CNC1]

The synthesis of 6-benzyl-6-azabicyclo[3.2.1]octane-2,7-dione is disclosed in Diaba, F. et al. Org. Lett. 2015, 17, 3860-3863. This compound can be used for the synthesis of compounds of Structure I as shown below.

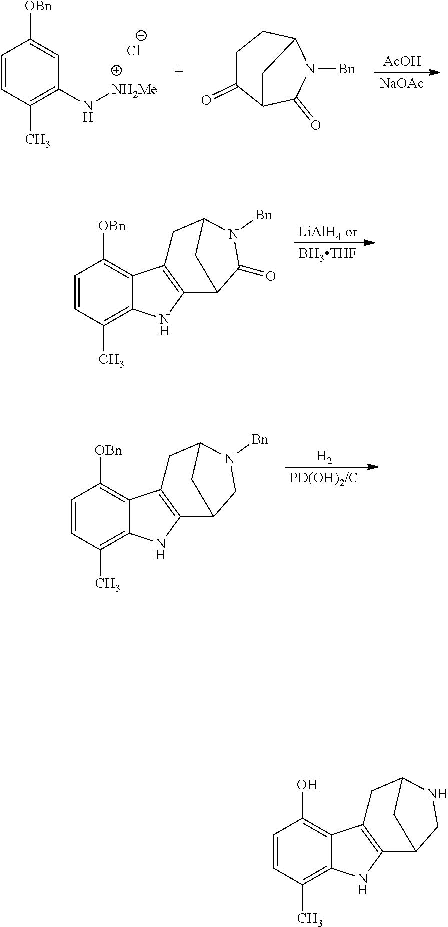

The synthesis of 3-(4-methoxyphenyl)-7-picolyl-7-azabicyclo[4.1.1]octane is disclosed in Zhao, J. et al. Org. Lett. 2017, 19, 4880-4883. Further transformations as shown below yield an azabicyclic ketone that can be used for the synthesis of compounds of Structure I.

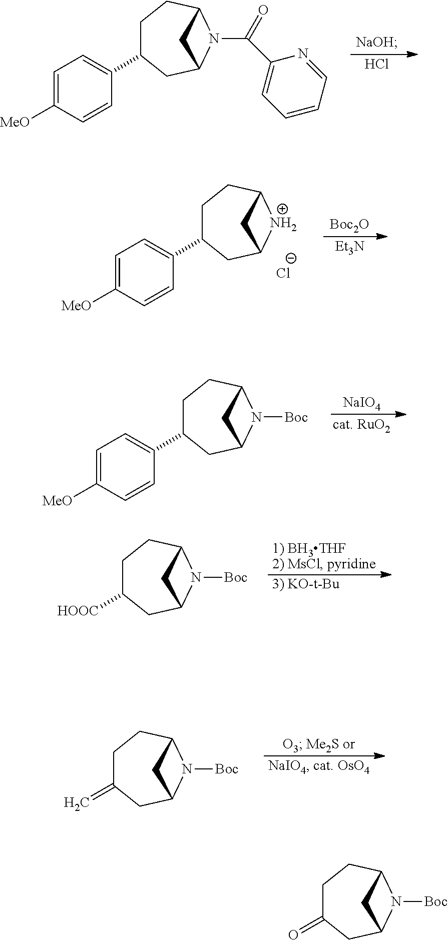

### Example 1. 7-Methyl-1,2,3,4,5,6-hexahydroazepino[4,5-b]indol-10-ol

### Step 1. 4-(Benzyloxy)-2-bromo-1-methylbenzene

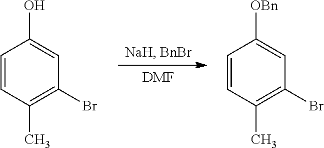

In a 500 mL 3-necked flask with stir bar, septum, dropping funnel with septum, Ar balloon, and ice bath was placed a suspension of NaH (60% in oil; 3.53 g, 88.2 mmol, 1.1 equiv.) in anhydrous DMF (20 mL). To this suspension was added dropwise with stirring and cooling in 15 min a solution of 3-bromo-4-methylphenol (15.0 g, 80.2 mmol) in anhydrous DMF (60 mL) to obtain an amber-colored suspension of the phenoxide. The mixture was stirred 15 min in the ice bath and another 15 min without cooling. After the mixture was re-cooled in an ice bath, BnBr (neat; 10.5 mL, 88.2 mmol, 1.1 equiv.) was added dropwise in 35 min. The mixture was stirred without temperature control for 2 h, then water (1 mL) was added cautiously. The solvent was distilled with gentle warming under an oil pump vacuum into a receiver cooled with dry ice. The residue was taken up in heptane (30 mL) and filtered with suction over celite from inorganic salts. The filter residue was washed with more heptane (2×20 mL), and the filtrate was evaporated. The evaporation residue was filtered over silica gel (11×7 cm, initially eluting with hexane, then with EtOAc/hexane 1:19). Impure late fractions were collected separately, evaporated, and resubjected to column chromatography (silica gel, 16×5.5 cm, hexane, then EtOAc/hexane 1:49). Again a small amount of impure late fractions was rejected. Essentially pure fractions from both columns were pooled and evaporated to yield 20.8 g (94%) of the benzyl ether as a nearly colorless oil. 1H NMR (CDCl₃, TMS) δ 7.43-7.36 (m, 4H), 7.35-7.30 (m, 1H), 7.19 (d, 1H, J=2.6 Hz), 7.12 (dd, 1H, J=8.4, 0.2 Hz), 6.84 (dd, 1H, J=8.4, 2.6 Hz), 5.02 (s, 2H), 2.32 (s, 3H).

### Step 2. tert-Butyl 2-[5-(Benzyloxy)-2-methylphenyl]-1-methylhydrazine-1-carboxylate

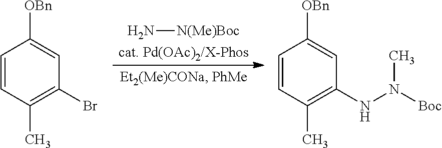

In a 100 mL 3-necked flask with stir bar, glass stopper, septum, reflux condenser with Ar balloon, and heating mantle was placed a suspension of NaH (60% in oil; 0.54 g, 13.6 mmol, 1.4 equiv.) in toluene (4 mL). The suspension was gently warmed, and 3-methyl-3-pentanol (2.15 mL, 17.5 mmol, 1.8 equiv.) was added in small portions over 20 min, resulting in hydrogen evolution. After another 10 min, a clear, brownish solution was obtained, which was allowed to cool to near rt. tert-Butyl 1-methylhydrazine-1-carboxylate (neat; 1.44 mL, 9.7 mmol) and 4-(benzyloxy)-2-bromo-1-methylbenzene (neat plus 0.5 mL toluene rinse; 2.69 g, 9.71 mmol) were added.

In the meantime, a solution of X-Phos (186 mg, 0.39 mmol, 40 mequiv.) in toluene (4 mL) was deoxygenated in a separate 3-necked flask by evacuating three times, followed each time by admission of argon. Palladium acetate (44 mg, 195 μmol, 20 mequiv.) was added to this solution through a temporarily opened side neck. The mixture was stirred at rt for 25 min to obtain a deep brownish-red catalyst solution, which was added by syringe as the last component to the above reaction mixture. The resulting dark-amber mixture was heated to reflux for 8 h. A precipitate began rapidly to form. After cooling, a thin layer chromatogram exhibited a major spot (silica gel, EtOAc/hexane 1:9 and 1:4: Rf approx. 0.2 and 0.5, respectively) besides several weak nonpolar spots and colored baseline material. Ethyl acetate/hexane 1:1 (60 mL) and water (50 mL) were added, the phases were separated, and the aqueous phase was extracted with ethyl acetate/hexane 1:1 (20 mL). The combined organic phases were concentrated, and m-xylene (10 mL) was added to the residue. The mixture was again evaporated to entrain the tertiary alcohol, resulting in a brown oil. This material was adsorbed on silica gel (12 g) and chromatographed on silica gel (16×5.5 cm, EtOAc/hexane 1:8). Evaporation of appropriate fractions yielded the amination product as a tan solid (2.33 g, 70%). 1H NMR (CDCl₃, TMS) δ 7.44-7.40 (m, 2H), 7.39-7.35 (m, 2H), 7.31 (m, 1H), 6.95 (dd, 1H, J=8.2, 0.4 Hz), 6.42 (dd, 1H, J=8.2, 2.5 Hz), 6.35 (d, 1H, J=2.5 Hz), 5.90 (br, 1H), 5.01 (s, 2H), 3.19 (s, 3H), 2.11 (s, 3H), 1.39 (br, 9H).

### Step 3. 1-[5-(Benzyloxy)-2-methylphenyl]-2-methylhydrazine hydrochloride

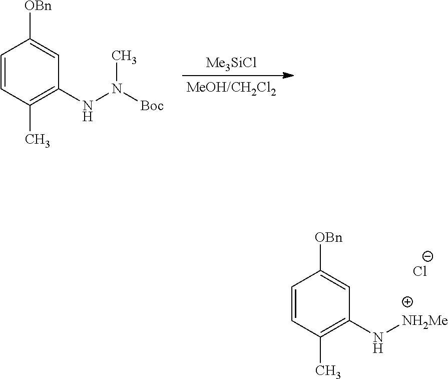

In a 150 mL round-bottom flask equipped with stir bar, septum, and Ar balloon (connected via a needle; for exclusion of moisture), the starting material (2.33 g, 6.80 mmol) was dissolved in a mixture of MeOH and CH₂Cl₂ (5 mL each). Neat chlorotrimethylsilane (1.30 mL, 10.2 mmol, 1.5 equiv.) was added in small portions with ice cooling over a period of 40 min. After another 20 min of stirring in the ice bath, the mixture consisted of a brown solution with a suspended light-colored solid. Stirring was continued at rt, whereon the precipitate initially dissolved but eventually reappeared. The reaction was followed by TLC on silica gel after aqueous workup of small aliquots (1M aqueous NaHCO₃/tert-BuOMe). With tert-BuOMe/hexane 1:2 as the mobile phase, the starting material exhibited an Rf of approx. 0.55, the product free base of approx. 0.15. After 5.2 h, toluene (10 mL) was added, and the more volatile solvents were removed by partial evaporation, leaving the product as a suspension in toluene. The product was filtered off with suction, washed with several small portion of toluene, and dried under vacuum to obtain 1.54 g (81%) of a faintly pinkish powder. 1H NMR (DMSO-d₆, TMS) δ 11.11 (br s, 2H), 7.83 (br s, 1H), 7.47-7.43 (m, 2H), 7.42-7.37 (m, 2H), 7.33 (m, 1H), 7.04 (d, 1H, J=8.5 Hz), 6.87 (d, 2H, J=2.3 Hz), 6.58 (dd, 1H, J=8.3, 2.4 Hz), 5.08 (s, 2H), 2.84 (s, 3H), 2.11 (s, 3H).

### Step 4. Benzyl 4-(Benzyloxy)-7-methylspiro[indole-3,4′-piperidine]-1′-carboxylate

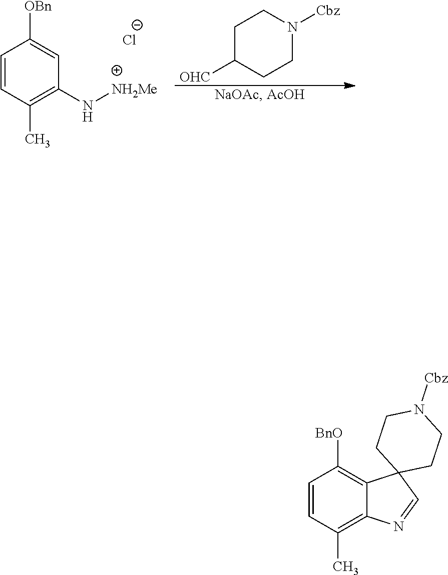

In a 100 mL round-bottom flask with stir bar and Ar balloon, a mixture of 1-[5-(benzyloxy)-2-methylphenyl]-2-methylhydrazine hydrochloride (522 mg, 1.87 mmol), N-Cbz-piperidine-4-carboxaldehyde (463 mg, 1.87 mmol), acetic acid (5 mL), and anhydrous NaOAc (161 mg, 1.96 mmol, 1.05 equiv.) was stirred in a 60° C. oil bath for 4.2 h. After cooling, a thin layer chromatogram after aqueous workup of a small aliquot (2M aqueous Na₂CO₃/EtOAc) exhibited a single mobile spot (SiO₂, EtOAc/hexane 1:1 and 65:35: Rf approx. 0.25 and 0.45, respectively) besides baseline material. The bulk of the solvent was distilled off under vacuum, and to the residue were added 1M aqueous NaHCO₃ solution and EtOAc (30 mL each). The phases were separated, and the aqueous phase was further extracted with EtOAc (15 mL). The combined organic phases were dried over Na₂SO₄ and evaporated. The residue was rapidly filtered over a silica gel column (12×2.5 cm, EtOAc/hexane 2:1). Evaporation of appropriate fractions yielded 507 mg (61%) of an amber glass. 1H NMR (CDCl₃, TMS) o 8.52 (s, 1H), 7.38-7.33 (m, 8H), 7.33-7.27 (m, 2H), 7.07 (dd, 1H, J=8.4, 0.7 Hz), 6.72 (d, 1H, J=8.4 Hz), 5.18 (br, 1H), 5.12 (br s, 3H), 4.38 (br, 1H), 4.31 (br, 1H), 3.22 (br, 2H), 2.64 (br, 2H), 2.51 (s, 3H), 1.41 (br, 2H). 13C NMR (CDCl₃, TMS) δ 174.42, 155.42, 153.76, 152.85, 136.98, 136.71, 130.30, 128.79, 128.66 (2C), 128.51 (2C), 128.03, 127.92 (2C), 127.84, 126.83 (2C), 123.63, 110.41, 69.77, 67.24, 57.29, 42.77, 28.31 (broadened), 16.01; one aromatic C and one aliphatic C not observed.

### Step 5. Benzyl 10-(Benzyloxy)-7-methyl-1,4,5,6-tetrahydroazepino[4,5-b]indole-3 (2H)-carboxylate

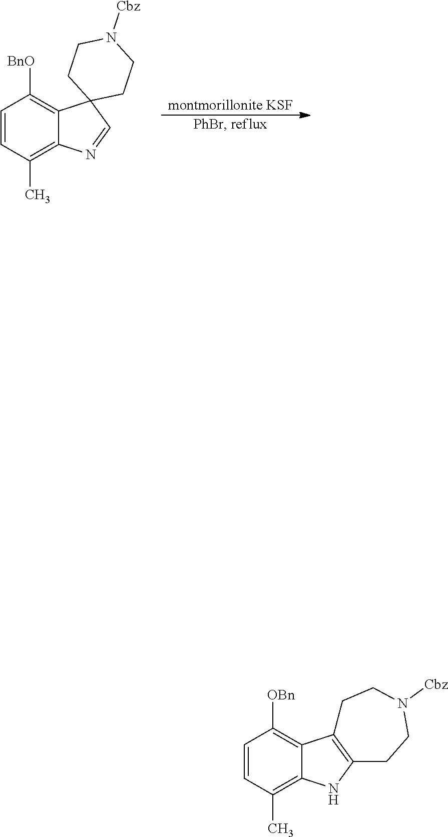 [SMILES: Cc1ccc(OCc2ccccc2)c2c1N=CC21CCN(C(=O)OCc2ccccc2)CC1 ; "PhBr, reflux" ; "montmorillonite KSF" ; Cc1ccc(OCc2ccccc2)c2c3c([nH]c12)CCN(C(=O)OCc1ccccc1)CC3]

In a 50 mL round-bottom flask with reflux condenser/Ar balloon, stir bar, and heating mantle, a mixture of benzyl 4-(benzyloxy)-7-methylspiro[indole-3,4′-piperidine]-1′-carboxylate (507 mg, 1.15 mmol), montmorillonite KSF clay (1.20 g), and bromobenzene (5 mL) was stirred at reflux temperature for 26.5, resulting in dark coloration of the clay. After cooling, a thin layer chromatogram exhibited a single defined spot (silica gel, EtOAc/hexane 1:1, Rf approx. 0.65). The mixture was filtered with suction over celite and the filter residue washed with CH₂Cl₂ (3×20 mL). The solution was partially evaporated to remove CH₂Cl₂, and the residual bromobenzene solution was chromatographed on silica gel (22×3.5 cm, EtOAc/hexane, initially 1:19 to remove bromobenzene, then 1:2). Appropriate eluate fractions were evaporated to obtain 369 mg (73%) of a tan solid. 1H NMR (CDCl₃, TMS; M=major, mi=minor urethane rotamer; ratio approx. 11:9) δ 7.68 (br, 1H, M), 7.62 (br, 1H, mi), 7.47-7.43 (m, 2H, M+mi), 7.41-7.28 (m, 8H, M+mi), 6.79 (d, 1H, too much overlap with the following to read J, mi), 6.78 (d, 1H, J=7.8 Hz, M), 6.46 (d, 1H, J=7.6 Hz, mi), 6.45 (d, 1H, J=7.7 Hz, M), 5.18 (s, 2H, M+mi), 5.14 (s, 2H, M), 5.13 (s, 2H, mi), 3.76 (m, 2H, mi), 3.70 (m, 2H, M), 3.42 (m, 2H, mi), 3.37 (m, 2H, M), 3.03 (m, 2H, M), 2.97 (m, 2H, mi), 2.36 (s, 3H, M+mi).

### Step 6. 7-Methyl-1,2,3,4,5,6-hexahydroazepino[4,5-b]indol-10-ol

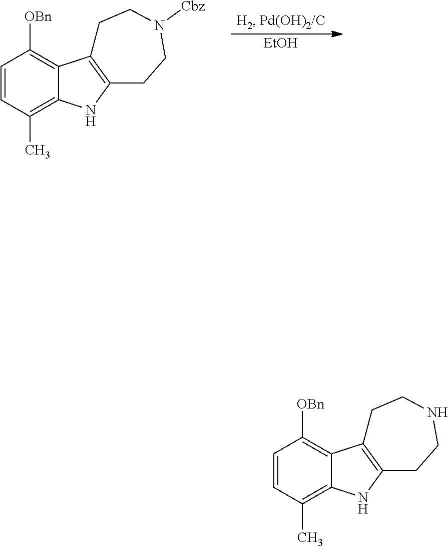 [SMILES: "EtOH" ; Cc1ccc(OCc2ccccc2)c2c3c([nH]c12)CCN(C(=O)OCc1ccccc1)CC3 ; "H2, Pd(OH)2/C" ; Cc1ccc(OCc2ccccc2)c2c3c([nH]c12)CCNCC3]

### Pharmacology

### Serotonin Receptor 5-HT₂ Functional Assays

Non-limiting examples of methods of measuring serotonin receptor functional activation are described as follows.

To measure serotonin receptor functional activation, either Gq dissociation by bioluminescence resonance energy transfer (BRET) or Gq-dependent calcium flux was performed for selected compounds. To measure 5-HT2 receptor-mediated Gq activation via Gq/γ1 dissociation as measured by BRET (McCorvy J D, Wacker D, Wang S, Agegnehu B, Liu J, Lansu K, Tribo A R, Olsen R H J, Che T, Jin J, Roth B L. Structural determinants of 5-HT2B receptor activation and biased agonism. Nat Struct Mol Biol. 2018; 25 (9): 787-96), HEK293T cells were sub-cultured in Dulbecco's Modified Eagle Medium (DMEM) supplemented with 10% dialyzed fetal bovine serum (FBS) and were co-transfected in a 1:1:1:1 ratio with RLuc8-fused human Gαq (Gαq-RLuc8), a GFP²-fused to the C-terminus of human Gγ1 (Gγ1-GFP²), human Gβ1, and 5-HT₂ receptor using TransiT-2020. After at least 18-24 hours, transfected cells were plated in poly-lysine coated 96-well white clear bottom cell culture plates in DMEM containing 1% dialyzed FBS at a density of 25,000-40,000 cells in 200 μL per well and incubated overnight. The next day, medium was decanted, and cells were washed with 60 μL of drug buffer (1×HBSS, 20 mM HEPES, pH 7.4), then 60 μL of drug buffer was added per well. Cells were pre-incubated in a humidified atmosphere at 37° C. before receiving drug stimulation. Drug stimulation utilized 30 μL addition of drug (3×) diluted in McCorvy buffer (1×HBSS, 20 mM HEPES, pH 7.4, supplemented with 0.3% BSA fatty acid free, 0.03% ascorbic acid), and plates were incubated for 1 hour at 37° C. Substrate addition occurred 15 minutes before reading and utilized 10 μL of the RLuc substrate coelenterazine 400a for Gq dissociation BRET² (Prolume/Nanolight, 5 μM final concentration). Plates were read for luminescence at 400 nm and fluorescent GFP² emission at 510 nm at 1 second per well using a Mithras L B940 (multimode microplate reader (e.g. one provided by Berthold)). The BRET ratios of fluorescence/luminescence were calculated per well and were plotted as a function of drug concentration using Graphpad Prism 8 (Graphpad Software Inc., San Diego, CA). Data were normalized to % 5-HT stimulation and analyzed using nonlinear regression “log (agonist) vs. response” to yield Eₘₐₓ and EC₅₀ parameter estimates.

Calcium flux was measured using stable-expressing 5-HT₂ Flp-In 293 T-Rex Tetracycline inducible system by methods known in the art (e.g. Investigation of the Structure-Activity Relationships of Psilocybin Analogues, ACS Pharmacol. Transl. Sci. 2020, Publication Date: Dec. 14, 2020, https://doi.org/10.1021/acsptsci.0c00176). Cell lines were maintained in DMEM containing 10% FBS, 10 μg/mL Blasticidin, and 100 μg/mL hygromycin B. At least 20-24 hours before the assay, receptor expression was induced with tetracycline (2 μg/mL), and cells were seeded into 384-well poly-L-lysine-coated black plates at a density of 7,500 cells/well in DMEM containing 1% dialyzed FBS. On the day of the assay, the cells were incubated for 1 hour at 37° C. with Fluo-4 Direct dye (Invitrogen, 20 μL/well) reconstituted in drug buffer (20 mM HEPES-buffered HBSS, pH 7.4) containing 2.5 mM probenecid. Drug dilutions were prepared at 5× final concentration in McCorvy buffer (20 mM HEPES-buffered HBSS, 0.1% BSA, 0.01% ascorbic acid, pH 7.4). After dye load, cells were allowed to equilibrate to room temperature for 15 minutes, and then placed in a FLIPR^{TETRA} fluorescence imaging plate reader (Molecular Devices). The FLIPR^{TETRA} was programmed to read baseline fluorescence for 10 s (1 read/s), and afterward 5 μL of drug per well was added, and fluorescence was read for a total of 5-10 min (1 read/s). Fluorescence in each well was normalized to the average of the first 10 reads for baseline fluorescence, and then either maximum-fold peak increase over baseline or area under the curve (AUC) was calculated. Either peak or AUC was plotted as a function of drug concentration, and data were normalized to percent 5-HT stimulation. Data were plotted, and non-linear regression was performed using “log (agonist) vs. response” in Graphpad Prism 8 to yield Eₘₐₓ and EC₅₀ parameter estimates.

The functional activity of various compounds disclosed herein at each of the 5-HT2A, 5-HT2B, and 5-HT2C receptors was measured against and relative to the functional activity of 4-hydroxytryptamine at those receptors. A comparison of the functional activities is provided in Table 2 as follows.

TABLE 2

| | | 5-HT2A | | 5-HT2B | | 5-HT2C | |
|---|---|---|---|---|---|---|---|
| | | BRET | | BRET | | BRET | |
| Compound | Identification | EC₅₀ | Eff | EC₅₀ | Eff | EC₅₀ | Eff |
| — | — | (nM) | % | (nM) | % | (nM) | % |
| 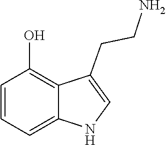 [SMILES: NCCc1c[nH]c2cccc(O)c12] | 4- hydroxytryptamine (reference compound) | 2.9 | 100 | 0.8 | 100 | 0.35 | 99 |
| 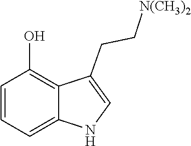 [SMILES: CN(C)CCc1c[nH]c2cccc(O)c12] | psilocin (reference compound) | 8.34 | 82 | 1.07 | 63 | 7.79 | 95 |
| 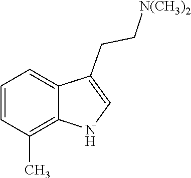 | 7,N,N-TMT (reference compound) | 232 | 80 | 74.3 | 36 | 11.7 | 79 |
| 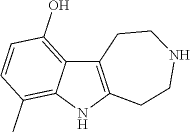 [SMILES: Cc1ccc(O)c2c3c([nH]c12)CCNCC3] | Cpd #1 (Pharm-136) | 0.6 | 102 | 3.5 | 52 | 0.5 | 101 |
| 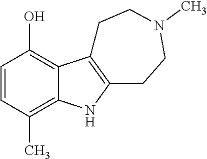 [SMILES: Cc1ccc(O)c2c3c([nH]c12)CCN(C)CC3] | Cpd #14 (W5) | 3.9 | 89 | 22.7 | 25 | 1.3 | 87 |
| 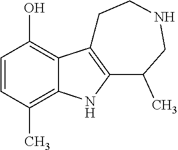 [SMILES: Cc1ccc(O)c2c3c([nH]c12)C(C)CNCC3] | Cpd #45 (W8) | 6.4 | 98 | 1.3 | 54 | 1.2 | 99 |
| 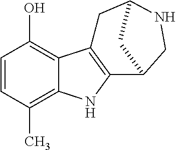 | Cpd #69 (W9) | — | — | — | — | — | — |
| 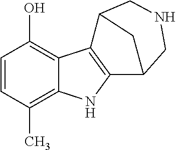 [SMILES: Cc1ccc(O)c2c3c([nH]c12)C1CNCC3C1] | Cpd #71 (W6) | — | — | — | — | — | — |
| 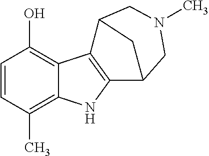 [SMILES: Cc1ccc(O)c2c3c([nH]c12)C1CC3CN(C)C1] | Cpd #72 (W7) | — | — | — | — | — | — |
| 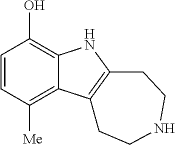 [SMILES: Cc1ccc(O)c2[nH]c3c(c12)CCNCC3] | (Pharm-159) | 3.5 | 96 | 3 | 95 | 0.17 | 99 |
| 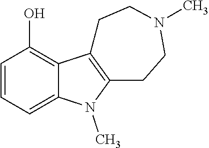 [SMILES: CN1CCc2c(n(C)c3cccc(O)c23)CC1] | Cpd #64 (A96) | 15.3 | 87 | 90.9 | 46 | 6.7 | 79 |
| 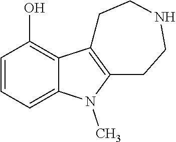 [SMILES: Cn1c2c(c3c(O)cccc31)CCNCC2] | Cpd #65 (A95) | 5.9 | 96 | 40.3 | 70 | 2.8 | 93 |
| 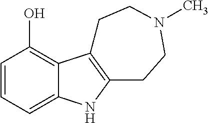 [SMILES: CN1CCc2[nH]c3cccc(O)c3c2CC1] | Cpd #66 (A94) | 8.1 | 91 | 47.9 | 28 | 18.8 | 89 |
| 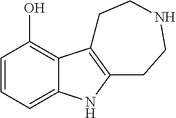 [SMILES: Oc1cccc2[nH]c3c(c12)CCNCC3] | Cpd #67 (A93) | 4 | 97 | 55.7 | 57 | 5.7 | 94 |
| 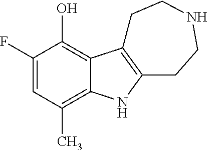 [SMILES: Cc1cc(F)c(O)c2c3c([nH]c12)CCNCC3] | Cpd #27 (A92) | 6.2 | 97 | 14 | 77 | 2.8 | 101 |
| 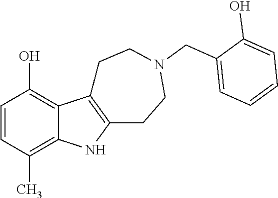 [SMILES: Cc1ccc(O)c2c3c([nH]c12)CCN(Cc1ccccc1O)CC3] | Cpd #18 (A90) | 31 | 76 | 2.7 | −2.6 | 24 | 93 |

Table 3 below summarizes the 5-HT receptor activity of psilocin and Compound #1 (also known as Pharm-136):

TABLE 3

| | |
|---|---|
| Psilocin | Compound Pharm-136 |

| | EC₅₀, nM | Eff, % | EC₅₀, nM | Eff, % |
|---|---|---|---|---|
| 5-HT_{1A} | 46.9 | 93 | 3997 | 73 |
| 5-HT_{1B} | 14.3 | 94 | 326.9 | 74 |
| 5-HT_{1D} | 2.2 | 102 | 325.6 | 72 |
| 5-HT_{1E} | 10.4 | 90 | 966.2 | 94 |
| 5-HT_{1F} | 9.3 | 93 | 460 | 56 |
| 5-HT_{2A} | 14.3 | 80 | 0.5 | 101 |
| 5-HT_{2B} | 2.1 | 32 | 4.2 | 50 |
| 5-HT_{2C} | 16.7 | 86 | 0.5 | 95 |

| | | | |
|---|---|---|---|
| 5-HT₄ | 562.6 | 81 | Not active |
| 5-HT_{5A} | 349 | 48 | Not active |

| | | | | |
|---|---|---|---|---|
| 5-HT₆ | 182.5 | 74 | 53.2 | 55 |

| | | | |
|---|---|---|---|
| 5-HT₇ | 524.5 | 86 | Not active |

Compounds of Formula 1 are generally known to potentially possess “hERG risk”. As a result of, many compounds disclosed in Table 1 and Table 2 were screened for the potential of “hERG risk” using pharmacological assays (e.g. Eurofins™ hERG Qube APC Assay). Compounds that exhibited strong potency at the 5-HT_{2A} receptor were further assessed on “hERG risk” based on comparative exposure/IC₅₀. A hERG IC₅₀ value that is 30-fold greater than therapeutic free plasma concentration is the threshold for deeming a compound to be of low “hERG risk”.

An important factor for hERG risk evaluation at early drug discovery stage is the hERG IC₅₀/in vitro 5-HT_{2A} ratio; compounds exhibiting a ratio of over 200 will exhibit a ratio of hERG IC₅₀ over estimated therapeutic plasma Cₘₐₓ of greater than 30 (which is generally indicative of low hERG risk potential at clinically relevant dose levels); compounds exhibiting a ratio between 150-200 are generally indicative of moderate hERG risk potential; compounds exhibiting a IC₅₀ (hERG)/EC₅₀ (5-HT 2A) ratio of less than or equal to 150 are generally indicative of high hERG risk potential.

Ames fluctuation data of compound 1 is reported in Table 4:

TABLE 4

| Bacterial | | Count | |
|---|---|---|---|
| strain | Concentration | (n. of wells) | Significance |
| TA98 − S9 | 5.0E−06M | 1 | Negative |
| | 1.0E−05M | 0 | Negative |
| | 5.0E−05M | 0 | Negative |
| | 1.0E−04M | 0 | Negative |
| TA98 + S9 | 5.0E−06M | 9 | Weak positive |
| | 1.0E−05M | 3 | Negative |
| | 5.0E−05M | 0 | Negative |
| | 1.0E−04M | 0 | Negative |
| TA100 − S9 | 5.0E−06M | 2 | Negative |
| | 1.0E−05M | 1 | Negative |
| | 5.0E−05M | 0 | Negative |
| | 1.0E−04M | 0 | Negative |
| TA100 + S9 | 5.0E−06M | 13 | Negative |
| | 1.0E−05M | 8 | Negative |
| | 5.0E−05M | 1 | Negative |
| | 1.0E−04M | 0 | Negative |
| TA1535 − S9 | 5.0E−06M | 0 | Negative |
| | 1.0E−05M | 0 | Negative |
| | 5.0E−05M | 0 | Negative |
| | 1.0E−04M | 0 | Negative |
| TA1535 + S9 | 5.0E−06M | 0 | Negative |
| | 1.0E−05M | 0 | Negative |
| | 5.0E−05M | 0 | Negative |
| | 1.0E−04M | 0 | Negative |
| TA1537 − S9 | 5.0E−06M | 0 | Negative |
| | 1.0E−05M | 1 | Negative |
| | 5.0E−05M | 0 | Negative |
| | 1.0E−04M | 0 | Negative |
| TA1537 + S9 | 5.0E−06M | 2 | Negative |
| | 1.0E−05M | 1 | Negative |
| | 5.0E−05M | 0 | Negative |
| | 1.0E−04M | 0 | Negative |

Various compounds disclosed herein were subjected to an in vitro SafetyScreen44™ panel for identifying off-target activity. Compounds of interest that are identified in the panel are then tested in follow up functional assays to identify if such compounds have a significant off-target risk. As summarized below in Table 5, compound 1 does not possess significant off-target activity. It is predicted that other compounds share similar characteristics with compound 1 and also lack of the off-target activity.

TABLE 5

| Compound | SafetyScreen44 ™ panel hits | Follow up studies |
|---|---|---|
| #1 (Pharm-136) | α1A(h) (antagonist radioligand) | No significant off- |
| | α2A(h) (antagonist radioligand | target activity |
| | β1(h) (agonist radioligand) | |
| | β2(h) (antagonist radioligand) | |
| | D2S(h) (agonist radioligand) | |
| | 5-HT1A(h) (agonist radioligand) | |
| | 5-HT1B (h) (antagonist radioligand) | |
| | 5-HT2A(h) (agonist radioligand) | |
| | 5-HT2B(h) (agonist radioligand) | |
| | dopamine transporter(h) (antagonist | |
| | radioligand) | |
| | COX2(h) | |

Additional experimentation was performed on compounds 1, W5, and A93. Table 6a, 6b and 6c summarize, among other things, the anticipated hepatic clearance of compounds 1, W5 and A93 respectively, in various species, as specified in Table 6a, 6b and 6c and as determined by calculating the in vitro hepatocyte clearance over time and scaling such rate with such species' hepatocyte count and the anticipated liver blood flow rate in such species.

TABLE 6a
Compound #1

| | | In vitro | | | |
|---|---|---|---|---|---|
| | | Clᵢₙₜ | | Hepatic | Predicted |
| | In vitro | (μL/min/ | Scale-up | Extraction | hepatic |
| | T_{1/2} | 10⁶ | Clᵢₙₜ | Ratio | CL |
| Species | (min) | cells) | (mL/min/kg) | (E) | (mL/min/kg) |
| Human | >511.69 | <2.71 | <6.89 | <0.25 | <5.17 |
| Monkey | >511.69 | <2.71 | <9.75 | <0.18 | <7.97 |
| Dog | >511.69 | <2.71 | <18.64 | <0.38 | <11.62 |
| Rat | 255.8 | 5.43 | 25.44 | 0.32 | 17.41 |
| Mouse | 42.2 | 32.95 | 389.27 | 0.81 | 73.10 |

TABLE 6b
Compound W5

| | | In vitro | | | |
|---|---|---|---|---|---|
| | | Clᵢₙₜ | | Hepatic | Predicted |
| | In vitro | (μL/min/ | Scale-up | Extraction | hepatic |
| | T_{1/2} | 10⁶ | Clᵢₙₜ | Ratio | CL |
| Species | (min) | cells) | (mL/min/kg) | (E) | (mL/min/kg) |
| Human | 1252.68 | 1.26 | 3.22 | 0.13 | 2.74 |
| Mouse | 34.08 | 40.67 | 480.45 | 0.84 | 75.80 |

TABLE 6c
Compound A93

| | | In vitro | | | |
|---|---|---|---|---|---|
| | | Clᵢₙₜ | | Hepatic | Predicted |
| | In vitro | (μL/min/ | Scale-up | Extraction | hepatic |
| | T_{1/2} | 10⁶ | Clᵢₙₜ | Ratio | CL |
| Species | (min) | cells) | (mL/min/kg) | (E) | (mL/min/kg) |
| Human | >511.69 | <2.71 | <6.89 | <0.25 | <5.17 |
| Mouse | >511.69 | <2.71 | <32.00 | <0.26 | <23.60 |

Tables 7a and 7b summarize, among other things, the observed plasma PK properties in animals, for Compound 1:

TABLE 7a

| | Route of | Dosing level | Clₜₒₜ | Vss | T_{1/2} | |
|---|---|---|---|---|---|---|
| Species | administration | (mg/kg) | [mL/h/Kg] | [mL/Kg] | (h) | AUCₗₐₛₜ |
| Rat | IV | 2.5 | 64319 | 204 | 3.93 | 37.5 |
| Dog | IV | 2 | 1476 | 5149 | 2.84 | 1198 |

TABLE 7b

| | Route of | Dosing level | Cl_F | Vz_F | T_{1/2} | |
|---|---|---|---|---|---|---|
| Species | administration | (mg/kg) | [mL/h/Kg] | [mL/Kg] | (h) | AUCₗₐₛₜ |
| Rat | IP | 10 | 48961 | 225480 | 3.30 | 193 |
| Dog | PO | 10 | NA | 7648 | 3.21 | 5465 |

Head Twitch Response (HTR) was assessed using a head-mounted neodymium magnet and a magnetometer detection coil, as described in Halberstadt et al., Psychopharmacology (Berl.), 2013, 227 (4): 727-739. Table 8 below summarizes the ED₅₀ in head twitch (as measured over a 60 minute interval) in mice as a result of Compound 1 as compared to psilocybin.

TABLE 8

| Cpd # | ED₅₀ in Head Twitch | |
|---|---|---|
| Psilocybin | 0.38 | mg/kg |
| Cpd #1 (Pharm-136) | 3.6 | mg/kg |

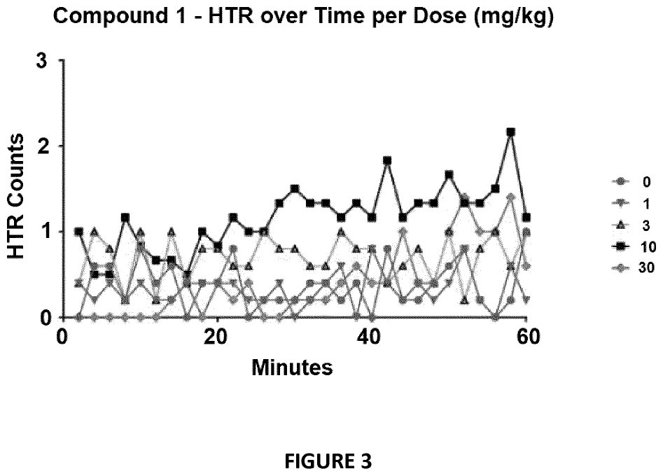

FIG. 3 illustrates the HTR relative to dose for Compound 1. Per FIG. 3, HTR over time per different doses of Compound 1 (as measured in mg/kg) are shown.

### Method of Use

Indole compounds described herein are believed to be useful in the treatment of drug resistant depression based on several clinical trials that have been reported using psilocybin itself.

A US STAR*D study has reported that more than half of all patients recruited through primary care and psychiatric clinics fail to achieve remission after first-line antidepressant treatment, and one-third were unable to experience remission after four courses of acute treatment (Rush A J, Trivedi M H, Wisniewski S R, Nierenberg A A, Stewart J W, Warden D, et al. Acute and longer-term outcomes in depressed outpatients requiring one or several treatment steps: a STAR*D report. Am. J. Psychiatry 2006; 163:1905-17).

In addition to the potential use of these analogs in the treatment of depression, other studies by third party groups of human volunteers have revealed that psilocybin can be used to treat tobacco and alcohol addiction. Moreover, in a controlled clinical environment, psilocybin was safely administered to subjects with OCD, and this drug treatment was found to lead to acute reductions in core OCD symptoms in several subjects (Moreno, F. A., Wiegand, C. B., Taitano, E. K., and Delgado, P. L. “Safety, tolerability, and efficacy of psilocybin in 9 patients with obsessive-compulsive disorder” J. Clin. Psychiatry 2006, 67, 1735-1740).

Another potential use of these analogs is in the treatment of seizure disorders, including but not limited to infantile seizure disorders such as but not limited to Dravet syndrome (Sourbon, J. et al. “Serotonergic Modulation as Effective Treatment for Dravet Syndrome in a Zebrafish Mutant Model”, ACS Chem. Neurosci. 2016, 7, 588-598).

The indole compounds described herein are believed to be safer than psilocybin, given their lack of at least some of the undesirable characteristics of 5-HT_{2B}-agonist related activities.

### Methods of Administration

As contemplated herein, a therapeutically effective amount of an indole compound described herein is administered to a subject in need thereof. Whether such treatment is indicated depends on the subject case, and is further subject to medical assessment (diagnosis) that takes into consideration signs, symptoms, and/or malfunctions that are present, the risks of developing particular signs, symptoms and/or malfunctions, and other factors.

As contemplated herein, an indole compound described herein may be administered by any suitable route known in the art. Such routes include, but are not limited to, oral, buccal, inhalation, topical, sublingual, rectal, vaginal, intracisternal or intrathecal through lumbar puncture, transurethral, nasal, percutaneous, transdermal, and parenteral administration (including intravenous, intramuscular, subcutaneous, intracoronary, intradermal, intramammary, intraperitoneal, intraarticular, intrathecal, retrobulbar, intrapulmonary injection and/or surgical implantation at a particular site). Parenteral administration may be accomplished using a needle and syringe or using a high pressure technique.

Pharmaceutical compositions include those wherein an indole compound described herein is present in a sufficient amount to be administered in an effective amount to achieve its intended purpose. The exact formulation, route of administration, and dosage is determined by a qualified medical practitioner in view of the diagnosed condition or disease. Dosage amount and interval can be adjusted individually to provide levels of an indole compound described herein that is sufficient to maintain the desired therapeutic effects. It is possible that the indole compound described herein may only require infrequent administration (e.g. monthly, as opposed to daily) to achieve the desired therapeutic effect.

As contemplated herein, a therapeutically effective amount of an indole compound described herein adapted for use in therapy varies with the nature of the condition being treated, the length of time that activity is desired, and the age and the condition of the patient, and ultimately is determined by the attendant physician. Dosage amounts and intervals can be adjusted individually to provide plasma levels of the indole compound that are sufficient to maintain the desired therapeutic effects. The desired dose conveniently may be administered in a single dose, or as multiple doses administered at appropriate intervals, for example as one, two, three, four, or more subdoses per day. Multiple doses often may be desired or required. For example, an indole compound described herein may be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days' rest, and another five daily doses (5/2/5); or, any dose regimen determined to be appropriate for the circumstance.

As contemplated herein, the indole compounds described herein may be administered in admixture with a pharmaceutical carrier selected with regard to the intended route of administration and standard pharmaceutical practice. Pharmaceutical compositions for use in accordance with the indole compounds described herein are formulated in a conventional manner using one or more physiologically acceptable carriers comprising excipients and auxiliaries that facilitate processing of the compounds described herein.

Water is a preferred carrier when an indole compounds described herein is administered intravenously. Saline solutions and aqueous dextrose and glycerol solutions may also be used as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The present compositions, if desired, can also contain minor amounts of wetting or emulsifying agents, or pH buffering agents.

These pharmaceutical compositions may be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of an indole compound described herein is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of an indole compound described herein. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a compound described herein.

When a therapeutically effective amount of an indole compound described herein described herein is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains an isotonic vehicle. An indole compound described herein described herein can be infused with other fluids over a 10-30 minute span or over several hours.

The indole compounds described herein may be readily combined with pharmaceutically acceptable carriers well-known in the art. Such carriers enable the active agents to be formulated as tablets, pills, dragees, capsules, liquids, gels, syrups, slurries, suspensions and the like, for oral ingestion by a patient to be treated.

Pharmaceutical preparations for oral use can be obtained by adding an indole compound described herein to a solid excipient, with or without grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

An indole compound described herein may be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of an indole compounds described herein can be prepared as appropriate oily injection suspensions. Suitable lipophilic solvents or vehicles include fatty oils or synthetic fatty acid esters. Aqueous injection suspensions can contain substances which increase the viscosity of the suspension.

In some embodiments, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

An indole compound described herein also may be formulated in rectal compositions, such as suppositories or retention enemas, e.g., containing conventional suppository bases. In addition to the formulations described previously, an indole compound described herein also can be formulated as a depot preparation. Such long-acting formulations can be administered by implantation (for example, subcutaneously or intramuscularly) or by intramuscular injection. Thus, for example, an indole compound described herein may be formulated with suitable polymeric or hydrophobic materials (for example, as an emulsion in an acceptable oil) or ion exchange resins.

An indole compound described herein may be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. The indole compounds described herein also may be injected parenterally, for example, intravenously, intramuscularly, subcutaneously, or intracoronarily. For parenteral administration, the indole compounds described herein may be best used in the form of a sterile aqueous solution which can contain other substances, for example, salts or monosaccharides, such as mannitol or glucose, to make the solution isotonic with blood. At least in some embodiments, indole compounds described herein are psilocybin analogs.

### General

It is contemplated that any part of any aspect or embodiment discussed in this specification may be implemented or combined with any part of any other aspect or embodiment discussed in this specification. While particular embodiments have been described in the foregoing, it is to be understood that other embodiments are possible and are intended to be included herein. It will be clear to any person skilled in the art that modification of and adjustment to the foregoing embodiments, not shown, is possible.

Unless defined otherwise, all technical and scientific terms used herein have the same meaning as is commonly understood by one of ordinary skill in the art to which this invention belongs. In addition, any citation of references herein is not to be construed nor considered as an admission that such references are prior art to the present invention.

The scope of the claims should not be limited by the example embodiments set forth herein, but should be given the broadest interpretation consistent with the description as a whole.

What is claimed is:

1. A chemical compound of Formula I, or any isotopologue or pharmaceutically acceptable salt thereof: 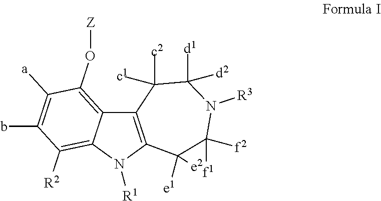
wherein:
R¹: (i) is selected from the group consisting of H, C₁-C₆ alkyl, C₁-C₆ substituted alkyl, C₂-C₆ alkenyl, C₂-C₆ alkynyl, C₃-C₆ cycloalkyl, (C₃-C₆ cycloalkyl) (C₁-C₆ alkyl), C₃-C₆ heterocyclyl, (C₃-C₆ heterocyclyl) (C₁-C₆ alkyl), aryl(C₁-C₆ alkyl), and heteroaryl(C₁-C₆ alkyl); or (ii) together with e¹ or e² form a chain of 2 to 4 carbon atoms to which are attached substituents independently selected from the group consisting of H, C₁-C₆ alkyl, aryl, heteroaryl, and any combination thereof; and
R²: (i) is selected from the group consisting of C₁-C₆ alkyl, C₁-C₆ substituted alkyl, C₂-C₆ alkenyl, C₂-C₆ alkynyl, C₃-C₆ cycloalkyl, (C₃-C₆ cycloalkyl) (C₁-C₆ alkyl), C₃-C₆ heterocyclyl, (C₃-C₆ heterocyclyl) (C₁-C₆ alkyl), aryl, aryl(C₁-C₆ alkyl), heteroaryl, heteroaryl(C₁-C₆ alkyl), CN, C(O)NH₂, C(O)NH(C₁-C₆ alkyl), C(O)N(C₁-C₃ alkyl) (C₁-C₆ alkyl), C(═NOH) (C₁-C₆ alkyl), and C(═NOH) (C₁-C₆ substituted alkyl), phenyl and halogen; or (ii) together with b form a chain of 2 or 3 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCF₃, cyano, and oxo; or (iv) is selected from the group consisting of H, C₁-C₆ alkyl, C₁-C₆ substituted alkyl, C₂-C₆ alkenyl, C₂-C₆ alkynyl, C₃-C₆ cycloalkyl, (C₃-C₆ cycloalkyl) (C₁-C₆ alkyl), C₃-C₆ heterocyclyl, (C₃-C₆ heterocyclyl) (C₁-C₆ alkyl), aryl, aryl(C₁-C₆ alkyl), heteroaryl, heteroaryl(C₁-C₆ alkyl), CN, C(O)NH₂, C(O)NH(C₁-C₆ alkyl), C(O)N(C₁-C₃ alkyl) (C₁-C₆ alkyl), C(═NOH) (C₁-C₆ alkyl), and C(═NOH) (C₁-C₆ substituted alkyl), if b is halogen, CH₃, CHF₂, CF₃, OCH₃, OCHF₂, OCF₃, SCH₃, SCHF₂, SCF₃, or cyano. In some embodiments, R² together with b form any one of CH₂CH₂, CH₂CH═CH₂, CH₂CH₂CH₂CH₂, CH═CHCH—CH, OCH₂CH₂, CH₂OCH₂, CH₂CH₂O, OCH═CH, CH═CHO, OCH₂O, SCH₂CH₂, CH₂SCH₂, CH₂CH₂S, SCH═CH, CH═CHS, NHCH₂CH₂, CH₂NHCH₂, CH₂CH₂NH, NHCH═CH, CH═CHNH, ON═CH, CH═NO, OCH═N, N═CHO, SN═CH, CH═NS, SCH═N, N═CHS, NHN═CH, CH═NNH, NHCH═N, N═CHNH, NHN═N, N═NNH, OCH₂CH₂CH₂, CH₂OCH₂CH₂, CH₂CH₂OCH₂, CH₂CH₂CH₂O, SCH₂CH₂CH₂, CH₂SCH₂CH₂, CH₂CH₂SCH₂, CH₂CH₂CH₂S NHCH₂CH₂CH₂, CH₂NHCH₂CH₂, CH₂CH₂NCH₂, CH₂CH₂CH₂NH, N═CHCH═CH, CH═NCH═CH, CH═CHN═CH, CH═CHCH═N. In some embodiments, R² together with b form any one of CH₂CH₂, CH₂CH₂CH₂, CH₂CH₂CH₂CH₂, CH═CHCH═CH, OCH₂CH₂, CH₂OCH₂, CH₂CH₂O, OCH═CH, CH═CHO, OCH₂O, SCH₂CH₂, CH₂SCH₂, CH₂CH₂S, SCH═CH, CH═CHS, NHCH═CH₂, CH₂NHCH₂, CH₂CH₂NH, NHCH═CH, CH═CHNH, ON═CH, CH═NO, OCH═N, N═CHO, SN═CH, CH═NS, SCH═N, N═CHS, NHN═CH, CH═NNH, NHCH═N, N═CHNH, NHN═N, N═NNH, OCH₂CH₂CH₂, CH₂OCH₂CH₂, CH₂CH₂OCH₂, CH₂CH₂CH₂O, SCH₂CH₂CH₂, CH₂SCH₂CH₂, CH₂CH₂SCH₂, CH₂CH₂CH₂S NHCH₂CH₂CH₂, CH₂NHCH₂CH₂, CH₂CH₂NCH₂, CH₂CH₂CH₂NH, N═CHCH═CH, CH═NCH—CH, CH═CHN═CH, CH—CHCH═N, wherein one hydrogen atom or two hydrogen atoms, if present on a moiety, are replaced with substituents selected independently from the group consisting of halogen, OH, C₁-C₆ alkoxy, C₁-C₆ alkyl, C₃-C₆ cycloalkyl, CHF₂, CF₃, OCHF₂, OCF₃, SCH₃, SCF₃, and cyano, or wherein two hydrogens, if attached to the same carbon atom, are replaced with an oxo group.

2. A chemical compound as claimed in claim 1, wherein:
a: (i) is selected from the group consisting of H, halogen, lower alkyl, CHF₂, CF₃, OCH₃, OCHF₂, OCF₃, SCHF₂, SCH₃, SCF₃, amine, and cyano; or (ii) together with Z form one of (A) a saturated chain of one oxygen and one carbon atom (with oxygen connected to the 5-position of the indole ring of Formula I), and (B) a chain of 2 or 3 carbon atoms, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo, and (C) a chain of 2 or 3 carbon atoms containing one double bond, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCHF₂, SCH₃, SCF₃, cyano, and oxo; or (iii) together with b form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo; and
b: (i) is selected from a group consisting of H, halogen, CH₃, CHF₂, CF₃, OCHs, OCHF₂, OCF₃, SCH₃, SCHF₂, SCF₃, amine, and cyano; or (ii) together with a form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo; or (iii) together with R² form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo.

3. The chemical compound as claimed in claim 1, wherein:
R³: (i) is selected from the group consisting of H, C₁-C₆ alkyl, C₂-C₆ alkenyl, C₂-C₆ alkynyl, C₃-C₆ cycloalkyl, (C₃-C₆ cycloalkyl) (C₁-C₆ alkyl), aryl(C₁-C₆ alkyl), acetyl, and heteroaryl(C₁-C₆ alkyl); or (ii) together with f and the N atom to which R³ is attached form an azetidine or pyrrolidine ring, such ring carrying substituents independently selected from the group consisting of H, aryl, heteroaryl, C₁-C₆ alkyl, and C₃-C₆ cycloalkyl; or (iii) together with d and the N atom to which R³ is attached form an azetidine or pyrrolidine ring, such ring carrying substituents independently selected from the group consisting of H, aryl, heteroaryl, halogen, C₁-C₆ alkyl, and C₃-C₆ cycloalkyl.

4. The chemical compound as claimed in claim 1, wherein:
Each of c, d, e, and f is H or a lower alkyl group; or c¹ and c² together form a part of a spiro-fused cyclopropane or cyclobutane ring, while each of d, e, and fare H or lower alkyl groups; or d¹ and d² together form a part of a spiro-fused cyclopropane or cyclobutane ring, while each of c, e, and/are H or lower alkyl groups; or e¹ and e² together form a part of a spiro-fused cyclopropane or cyclobutane ring, while each of c, d, and fare H or lower alkyl groups; or f and f together form a part of a spiro-fused cyclopropane or cyclobutane ring, while each of c, d, and e are H or lower alkyl groups; or one of c and one of d together form —CH₂— or —CH₂CH₂—, thereby giving rise to a condensed cyclopropane or cyclobutane ring, while the remainder of each of c, d, e, and f are H or lower alkyl groups; or one of e and one of/together form —CH₂— or —CH₂CH₂—, thereby giving rise to a condensed cyclopropane or cyclobutane ring, while the remainder of each of c, d, e, and f are H or lower alkyl groups; or one of c and one of e together form —CH₂— or —CH₂CH₂—, thereby giving rise to a bridged bicyclic substructure, while the remainder of each of c, d, e, and f are H or lower alkyl groups; or one of c and one of/together form —CH₂— or —CH₂CH₂—, thereby giving rise to a bridged bicyclic substructure, while the remainder of each of c, d, e, and f are H or lower alkyl groups; or one of d and one of e together form —CH₂— or —CH₂CH₂—, thereby giving rise to a bridged bicyclic substructure, while the remainder of each of c, d, e, and f are H or lower alkyl groups; or one of/and one of e together form —CH₂— or —CH₂CH₂—, thereby giving rise to a condensed bicyclic substructure, while the remainder of each of c, d, e, and f are H or lower alkyl groups; or e together with R¹ form a chain of 2 to 4 carbon atoms to which are attached substituents independently selected from the group consisting of H, C₁-C₆ alkyl, aryl, heteroaryl, and any combination thereof; or one of c and R³ together form —CH₂— or —CH₂CH₂—, thereby giving rise to a bridged bicyclic substructure, while the remainder of each of c, d, e, and f are H or lower alkyl groups; or one of d and R³ together form —CH₂— or —CH₂CH₂—, thereby giving rise to a condensed bicyclic substructure, while the remainder of each of c, d, e, and f are H or lower alkyl groups; or one of e and R³ together form —CH₂— or —CH₂CH₂—, thereby giving rise to a condensed bicyclic substructure, while the remainder of each of c, d, e, and f are H or lower alkyl groups; or one of f and R³ together form —CH₂— or —CH₂CH₂—, thereby giving rise to a bridged bicyclic substructure, while the remainder of each of c, d, e, and f are H or lower alkyl groups.

5. The chemical compound as claimed in claim 1, wherein:
Z: (i) is selected from the group consisting of H, R⁵, (R⁶) (R⁷)N—C(O)—, C₁-C₆ alkyl-C(O), C₃-C₆ cycloalkyl-C(O), aryl-C(O), and heteroaryl-C(O), wherein R⁵ is selected from the group consisting of C₁-C₆ alkyl, C₂-C₆ alkenyl, C₂-C₆ alkynyl, C₃-C₆ cycloalkyl, (C₃-C₆ cycloalkyl) (C₁-C₆ alkyl), aryl(C₁-C₆ alkyl), and heteroaryl(C₁-C₆ alkyl), and wherein R⁶ and R⁷ are each independently selected from the group consisting of H, C₁-C₄ alkyl, and C₃-C₆ cycloalkyl or are joined to form a 4-7 membered heterocyclyl group; or (ii) is (R⁸O) (R⁹O) P(O)—, wherein R⁸ and R⁹ are each independently H or a cationic counterion of a phosphate salt form such as sodium, potassium, one-half of magnesium, one-half of calcium, ammonium, or ammonium substituted with one or more alkyl or cycloalkyl groups; or (iii) together with c form a linkage that gives rise to a pyran or oxepan ring comprising substituents independently selected from the group consisting of H, halogen, C₁-C₆ alkyl, and C₃-C₆ cycloalkyl; or (iv) together with a form one of (A) a saturated chain of one oxygen and one carbon atom (with oxygen connected to the 5-position of the indole ring of Formula I), and (B) a chain of 2 or 3 carbon atoms, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo, and (C) a chain of 2 or 3 carbon atoms containing one double bond and carrying substituents independently selected from the group consisting of H, halogen, OH, C₁-C₆ alkoxy, C₁-C₆ alkyl, C₅-C₆ cycloalkyl, CHF₂, CF₃, OCHF₂, OCF₃, SCH₃, SCHF₂, SCF₃, cyano, and oxo.

6. The chemical compound as claimed in claim 1, wherein: (i) R¹ is selected from the group consisting of H, C₁-C₆ alkyl, and C₁-C₆ substituted alkyl; (ii) R² is selected from the group consisting of C₁-C₆ alkyl, and C₁-C₆ substituted alkyl; (iii) each of a and b is selected from the group consisting of H, halogen, lower alkyl, CHF₂, CF₃, OCH₃, OCHF₂, OCF₃, SCHF₂, SCH₃, SCF₃, and cyano; (iv) each of c, d, e, and f is H or a lower alkyl group; and (v) R³ is selected from the group consisting of H and C₁-C₆ alkyl.

7. The chemical compound as claimed in claim 1, wherein the compound has the following formula:

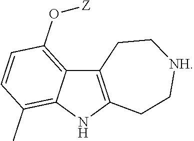

8. The chemical compound as claimed in claim 1, wherein the compound is 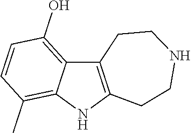 [SMILES: Cc1ccc(O)c2c3c([nH]c12)CCNCC3]
or the conjugate base thereof.

9. The chemical compound as claimed in claim 1, wherein the compound is 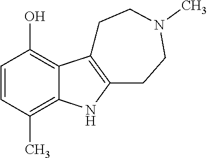 [SMILES: Cc1ccc(O)c2c3c([nH]c12)CCN(C)CC3]
or the conjugate base thereof.

10. The chemical compound as claimed in claim 1, wherein the compound is 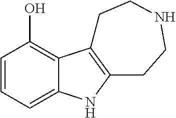 [SMILES: Oc1cccc2[nH]c3c(c12)CCNCC3]
or the conjugate base thereof.

11. A method of treating a disorder comprising administering to a patient an effective amount of the compound as claimed in claim 1.

12. The method as claimed in claim 11, wherein the disorder is selected from the group consisting of major depressive disorder, drug resistant depression, and psychotic depression, addiction including alcoholism, tobacco addiction, cocaine addiction, and opioid addiction, pain indications including neuropathic pain, pain from chemotherapy associated neuropathy, phantom limb pain and fibromyalgia, inflammation (including chronic and acute), eating disorders including anorexia, autism, cluster headaches, migraines, dementia including Alzheimer's dementia, Parkinson's disease dementia, and Lewy body dementia, post-traumatic stress disorder, emotional distress associated with cancer, Fragile-X syndrome, autism spectrum disorder, bipolar disease, obsessive compulsive disease, and Rett syndrome.

13. A method of treating a disorder comprising administering to a patient an effective amount of the compound as claimed in claim 8.

14. The method as claimed in claim 13, wherein the disorder is selected from the group consisting of major depressive disorder, drug resistant depression, and psychotic depression, addiction including alcoholism, tobacco addiction, cocaine addiction, and opioid addiction, pain indications including neuropathic pain, pain from chemotherapy associated neuropathy, phantom limb pain and fibromyalgia, inflammation (including chronic and acute), eating disorders including anorexia, autism, cluster headaches, migraines, dementia including Alzheimer's dementia, Parkinson's disease dementia, and Lewy body dementia, post-traumatic stress disorder, emotional distress associated with cancer, Fragile-X syndrome, autism spectrum disorder, bipolar disease, obsessive compulsive disease, and Rett syndrome.

15. Use of the compound as claimed in claim 8 in the treatment of a disorder selected from the group consisting of major depressive disorder, drug resistant depression, and psychotic depression, addiction including alcoholism, tobacco addiction, cocaine addiction, and opioid addiction, pain indications including neuropathic pain, pain from chemotherapy associated neuropathy, phantom limb pain and fibromyalgia, inflammation (including chronic and acute), eating disorders including anorexia, autism, cluster headaches, migraines, dementia including Alzheimer's dementia, Parkinson's disease dementia, and Lewy body dementia, post-traumatic stress disorder, emotional distress associated with cancer, Fragile-X syndrome, autism spectrum disorder, bipolar disease, obsessive compulsive disease, and Rett syndrome.

16. The use as claimed in claim 15, wherein the disorder is selected from the group consisting of major depressive disorder, drug resistant depression, and psychotic depression.
